SEQUENCE LISTING (10 sequences)

SEQ ID NO: 1 - DNA, 10088 bp, Homo sapiens
  misc_feature (1)..(10088): Nucleotide sequence for human LRP6
gtgcccctttctttcttctctcgctgggaagctgggaagtatgagcgtgcagccctgccgctgcggcggccgccccggctcctcgcctcccccacttctggccacccctcgccggtgagagaagagaacgcgagaagggaagatgggggccgtcctgaggagcctcctggcctgcagcttctgtgtgctcctgagagcggcccctttgttgctttatgcaaacagacgggacttgcgattggttgatgctacaaatggcaaagagaatgctacgattgtagttggaggcttggaggatgcagctgcggtggactttgtgtttagtcatggcttgatatactggagtgatgtcagcgaagaagccattaaacgaacagaatttaacaaaactgagagtgtgcagaatgttgttgtttctggattattgtcccccgatgggctggcatgtgattggcttggagaaaaattgtactggacagattctgaaactaatcggattgaagtttctaatttagatggatctttacgaaaagttttattttggcaagagttggatcaacccagagctattgccttagatccttcaagtgggttcatgtactggacagactggggagaagtgccaaagatagaacgtgctggaatggatggttcaagtcgcttcattataataaacagtgaaatttactggccaaatggactgactttggattatgaagaacaaaagctttattgggcagatgcaaaacttaatttcatccacaaatcaaatctggatggaacaaatcggcaggcagtggttaaaggttcccttccacatccttttgccttgacgttatttgaggacatattgtactggactgactggagcacacactccattttggcttgcaacaagtatactggtgagggtctgcgtgaaatccattctgacatcttctctcccatggatatacatgccttcagccaacagaggcagccaaatgccacaaatccatgtggaattgacaatgggggttgttcccatttgtgtttgatgtctccagtcaagcctttttatcagtgtgcttgccccactggggtcaaactcctggagaatggaaaaacctgcaaagatggtgccacagaattattgcttttagctcgaaggacagacttgagacgcatttctttggatacaccagattttacagacattgttctgcagttagaagacatccgtcatgccattgccatagattacgatcctgtggaaggctacatctactggactgatgatgaagtgagggccatacgccgttcatttatagatggatctggcagtcagtttgtggtcactgctcaaattgcccatcctgatggtattgctgtggactgggttgcacgaaatctttattggacagacactggcactgatcgaatagaagtgacaaggctcaatgggaccatgaggaagatcttgatttcagaggacttagaggaaccccgggctattgtgttagatcccatggttgggtacatgtattggactgactggggagaaattccgaaaattgagcgagcagctctggatggttctgaccgtgtagtattggttaacacttctcttggttggccaaatggtttagccttggattatgatgaaggcaaaatatactggggagatgccaaaacagacaagattgaggttatgaatactgatggcactgggagacgagtactagtggaagacaaaattcctcacatatttggatttactttgttgggtgactatgtttactggactgactggcagaggcgtagcattgaaagagttcataaacgaagtgcagagagggaagtgatcatagatcagctgcctgacctcatgggcctaaaggctacaaatgttcatcgagtgattggttccaacccctgtgctgaggaaaacgggggatgtagccatctctgcctctatagacctcagggccttcgctgtgcttgccctattggctttgaactcatcagtgacatgaagacctgcattgtcccagaggctttccttttgttttcacggagagcagatatcagacgaatttctctggaaacaaacaataataatgtggctattccactcactggtgtcaaagaagcttctgctttggattttgatgtgacagacaaccgaatttattggactgatatatcactcaagaccatcagcagagcctttatgaatggcagtgcactggaacatgtggtagaattcggcttagattatccagaaggcatggcagtagactggcttgggaagaacttgtactgggcagacacaggaacgaatcgaattgaggtgtcaaagttggatgggcagcaccgacaagttttggtgtggaaagacctagatagtcccagagctctcgcgttggaccctgccgaaggatttatgtattggactgaatggggtggaaaacctaagatagacagagctgcaatggatggaagtgaacgtactaccttagttccaaatgtggggcgggcaaacggcctaactattgattatgctaaaaggaggctttattggacagacctggacaccaacttaatagaatcttcaaatatgcttgggctcaaccgtgaagttatagcagatgacttgcctcatccttttggcttaactcagtaccaagattatatctactggacggactggagccgacgcagcattgagcgtgccaacaaaaccagtggccaaaaccgcaccatcattcagggccatttggattatgtgatggacatcctcgtctttcactcatctcgacagtcagggtggaatgaatgtgcttccagcaatgggcactgctcccacctctgcttggctgtgccagttgggggttttgtttgtggatgccctgcccactactctcttaatgctgacaacaggacttgtagtgctcctacgactttcctgctcttcagtcaaaagagtgccatcaaccgcatggtgattgatgaacaacagagccccgacatcatccttcccatccacagccttcggaatgtccgggccattgactatgacccactggacaagcaactctattggattgactcacgacaaaacatgatccgaaaggcacaagaagatggcagccagggctttactgtggttgtgagctcagttccgagtcagaacctggaaatacaaccctatgacctcagcattgatatttacagccgctacatctactggacttgtgaggctaccaatgtcattaatgtgacaagattagatgggagatcagttggagtggtgctgaaaggcgagcaggacagacctcgagccgttgtggtaaacccagagaaagggtatatgtattttaccaatcttcaggaaaggtctcctaaaattgaacgggctgctttggatgggacagaacgggaggtcctctttttcagtggcttaagtaaaccaattgctttagcccttgatagcaggctgggcaagctcttttgggctgattcagatctccggcgaattgaaagcagtgatctctcaggtgctaaccggatagtattagaagactccaatatcttgcagcctgtgggacttactgtgtttgaaaactggctctattggattgataaacagcagcaaatgattgaaaaaattgacatgacaggtcgagagggtagaaccaaagtccaagctcgaattgcccagcttagtgacattcatgcagtaaaggagctgaaccttcaagaatacagacagcacccttgtgctcaggataatggtggctgttcacatatttgtcttgtaaagggggatggtactacaaggtgttcttgccccatgcacctggttctacttcaagatgagctatcatgtggagaacctccaacatgttctcctcagcagtttacttgtttcacgggggaaattgactgtatccctgtggcttggcggtgcgatgggtttactgaatgtgaagaccacagtgatgaactcaattgtcctgtatgctcagagtcccagttccagtgtgccagtgggcagtgtattgatggtgccctccgatgcaatggagatgcaaactgccaggacaaatcagatgagaagaactgtgaagtgctttgtttaattgatcagttccgctgtgccaatggtcagtgcattggaaagcacaagaagtgtgatcataatgtggattgcagtgacaagtcagatgaactggattgttatccgactgaagaaccagcaccacaggccaccaatacagttggttctgttattggcgtaattgtcaccatttttgtgtctggaactgtatactttatctgccagaggatgttgtgtccacgtatgaagggagatggggaaactatgactaatgactatgtagttcatggaccagcttctgtgcctcttggttatgtgccacacccaagttctttgtcaggatctcttccaggaatgtctcgaggtaaatcaatgatcagctccctcagtatcatggggggaagcagtggacccccctatgaccgagcccatgttacaggagcatcatcaagtagttcttcaagcaccaaaggcacttacttccctgcaattttgaaccctccaccatccccagccacagagcgatcacattacactatggaatttggatattcttcaaacagtccttccactcataggtcatacagctacaggccatatagctaccggcactttgcaccccccaccacaccctgcagcacagatgtttgtgacagtgactatgctcctagtcggagaatgacctcagtggcaacagccaagggctataccagtgacttgaactatgattcagaacctgtgcccccacctcccacaccccgaagccaatacttgtcagcagaggagaactatgaaagctgcccaccttctccatacacagagaggagctattctcatcacctctacccaccgccaccctctccctgtacagactcctcctgaggaggggccctcctcctctgactgcctccaacgtaaaaatgtaaatataaatttggttgagatctggagggggggagggagctattagagaaggatgaggcagaccatgtacagttaaaattataaaatggggtagggaatactggagatatttgtacagaagaaaaggatatttatatattttcttaaaacagcagatttgctgcttgtgccataaaagtttgtataaaaaaaatttgtactaaaagttttatttttgcaaactaaatacacaaagcatgccttaaacccagtgaagcaactgagtacaaaggaaacaggaataataaaggcatcactgaccaggaatatctgggctttattgataccaaaaataaaaaagaggaagaagaaaaattaagtccatctcagagcagcaaaccatagatacatggatgtagccagatagccttcagttaactaacatttgagggccaacaagtaagaaatgatgaaaggaaaaaaatgcaattaatactaaccttggacgaagggctttgttttctctaggaatccaacagtgctagtgaggaaagtagatatttctaaaaacccattctgggtgttgctgttgtaggagagatcagccctctggtaagatgccatgaagctgtgtgtgtgtgcaagtctctgtccctacctttagaatccatacctctgtcaaaatgaatttttttctctaggtatgtttaccttgctgcctcctccagcaacttggtaagtcattttgctaagataccatgatttttttaagctgaagcattgactaaatggaattttctaaattaaacttgattttaatatttcttctagctccattccccagtaggcttagctcttcaatttgactgctgtttttgcataatgatcaaaagttagacatattatttctcttcttccaagattgttttaatgctcattaaaatgtctttttacaacacatatagacaatgtttaagaattaaaaatttaaccattatgtttttgttgtaaatctcatatccttgcactactttcagcatatatcacagtacgaaatcatttatatatatatatatatatatatatatatatatatatatatatatatatatattttgtttgtttgtttgttttctgagtaaaacatttaaatatgttctggttagagacaatctatttaaaaagatttttttcttattaggattttccctatattaacagtttgtgatgttttcatgttctttagaccggtttttctcagaataatgtctacatacatacctcttctaatgtgtgacatgaatttaatatctttctgttacccactgtgaatgttaggctgttttcaaattatccacaaattattcttgtaatcacccaatatttttatgtgggtcctctcttacccattatggattaagatagtttaacaaatttaacaatgaggattaaatgagaaggcaaactgttaacttctcagctgtcagaatttgggtggaagggaataatggaagcctcttttgtgatctgcctgacctgctgtcatgtatggtactggggctgctacatcttgagctatcagggctgacctgtggaatgattctagcacttgctctgccaccttgccagaagttcgtttcctgctttttacacatgtgtagcacttctctgctaaaattgaatggttttaaactaatgtatttttagcttaagaggtgttggtcagttaattattgaattttttttttttcttttttaattctgtcttgccaaggcctctctgggtttcagggcccaagagaaaacagtggaagaaaggattcagaatttgggcaagggtgaagtaactgttcatgcaagttaaaaatacctaagtaaagtttttgaagataaaattgtggtttcagaataatgctgattgttggagactgtaagaatcaggtgcacttgattttgcatataagcaaatggtaaatctatcagaatcctaaaacagacaagcatgaactcttcccattgctggaactaagtgcccacagtgtcagacaaaatggacattgaacttggattctgtgatacacagggcacttgatgcttaaatgaagatggaaaggttagcaatacctgggtgtcagttagaatttgagaattctatatgtttacatatttaaatgtgcatcttgatctggtgggcttcccatgtggagacttgcactctaattaactaagaagaatattgccttgttggatctcagtccacgtgcttgcactgcgatggcaatggcctcttcttcaaaatactaatttgtgtgccaatttgtttaaaattatttgaaggcagttcagcctaatctcagtgttctctttctggggtagatgagatggattcttaatatttctgggagtactttttaatgagagaattgtcaaatttggaaagatttattgagccttaggttacatggacagttaagcttaagtaaactgtatattgattatcaaacacaagctgtaattggaaaagttgagaggaaaagcatgagatcacaaattagggggaaaaaagaaaagggatttttaaatttggtgtattaaattcattgtccaagggggaaaatgaataatgtttcattagattccttatatgcaaaagtatttattttgaacatgtgtcctaaaatatatgcactaactgatgtgattaaaattgtccaagaaataaacttgagcataacatactttgtgtgcaccacagtaagctattctgcattgaagtggtcttttataactaaggcctggactttgctccaacagagtcgtggtcttctgaatagtgacttaaggagttttgtttgcttaagtcagataatagcacattcacagggaaacaaagagagttggtggatagaattttctgactattaatttttcttccatgaaattttattatgcctttggcactttctgccactcttacagcatatcacaagatatctgtttagcagaagattatgtagttactttaattttaatataaaagtagcttgtgatacattaccaagagatctctgattctttagtaagtttgagaacacctattctacagagatgataggtacttagaaatgaagactttaaagtacattttaatctaatataggccagtaattgggggaaggggctttgagcagtacaattttaagatgattttgagggttgtatttctttatcatttaaaaatatcctaaagtcagtaatttatatgaaggaaactcattcattattgaaggtattaaaaatagccatcatctgtattaggtagcagttttggaggatcatctttttcttttgctataaagccctattaatgaagaatacttccagtagagttaatagctgtagcttacctagtgtgttaatgaagtgtgtttatttatgtgacttgataccagtagtcataatagagactgaagaggtatgcgttaagcacgcctacttctatgcagtaaacaggctgcagctgcctagattagattcttagaaatgtcatattttgaattgttttatttcttgtaggggaagctttgtcccacttcattcatttgcatgccataggaattacatattggttatcattacgtatctaacaagattcagaaacaaaaatcttggacttttcacatccgaaatatgtcagctcttaataaatgtgtggtgcttaagtctacatatggcatccatagttgatttagagtatggatatgagtgtgttgaccagttatcagtaggtggacaaatatttgggcatctacagatgagactatgcactaagtgtggactgagtcctaaagaagcttatagtcaggtgttgtttaaaacattatcagaattcttaaacccaaggaatttaattttatttggtatttcttaagcctaaaatgaaccaagagaaagatgattttagaaagtacttgtagtgaaagatgattttagaaagtacttgtagtgcatgtgtggcttctgacttttgggatggcaccattttataatagtttcaaaatttagcttttgaaattctcaacattttatggtagaagactttggacctcaagtataaaattatacgtttataatttttttaaaatttaaattataagtattgtgaattcacactctcaggctattgtctgacttgatctacgtctcataaagcctgtacctgagtggagtggaaggtggagtcttaggttaatcagttactgactctaccctcaccctctttcaattgaggtaaactttgctgtttttctttttcataaagcattctcaaattgttgagtttattgctgaaaaaaatctccatgactttacagatagaattacaaactaaatgatgtcttgtatttagaagcagagtacagacctaacgaactgttagattctccaccatcacttagggtttgcccagaagcaacaccagagaattacagacaacgcgcttttgctgaactgtccattttggtggttgtgtttttcagtcaaatataagcaggatgggcgatagagatatatttatatatagatacatattctatatatctaatgcctaaatatgggtattaaagggaaaatttttaaagtctgattaaatccaatatgacatgaaattaaatatatggattagtaaggaaaaatgttaaaaagtagagaggataccaagaagattaaactggactagccttatttgcaagtgaaggatctggtgctgctttcagatgtttatcttttatttttttcccttaagctttaatcttcgtcattgtcttaaagtcaactggtgtttcttgttcattgactttggtacgatggtgctttgcaaggatgtatttatgttataatggccaacatttggtcagcccttgtccacttattcacttccctccttttgtaaaataagtgctttaattataaactgtataaaaataccttgtataaaccccttttttgattattacaataaataagctgaattgtaacaaatgaaatttgatttttgtaataaaacagtggaaaagtaaaaaaa

SEQ ID NO: 2 - protein, 1613 aa, Homo sapiens
  MISC_FEATURE (1)..(1613): Amino acid sequence for human LRP6
MGAVLRSLLACSFCVLLRAAPLLLYANRRDLRLVDATNGKENATIVVGGLEDAAAVDFVFSHGLIYWSDVSEEAIKRTEFNKTESVQNVVVSGLLSPDGLACDWLGEKLYWTDSETNRIEVSNLDGSLRKVLFWQELDQPRAIALDPSSGFMYWTDWGEVPKIERAGMDGSSRFIIINSEIYWPNGLTLDYEEQKLYWADAKLNFIHKSNLDGTNRQAVVKGSLPHPFALTLFEDILYWTDWSTHSILACNKYTGEGLREIHSDIFSPMDIHAFSQQRQPNATNPCGIDNGGCSHLCLMSPVKPFYQCACPTGVKLLENGKTCKDGATELLLLARRTDLRRISLDTPDFTDIVLQLEDIRHAIAIDYDPVEGYIYWTDDEVRAIRRSFIDGSGSQFVVTAQIAHPDGIAVDWVARNLYWTDTGTDRIEVTRLNGTMRKILISEDLEEPRAIVLDPMVGYMYWTDWGEIPKIERAALDGSDRVVLVNTSLGWPNGLALDYDEGKIYWGDAKTDKIEVMNTDGTGRRVLVEDKIPHIFGFTLLGDYVYWTDWQRRSIERVHKRSAEREVIIDQLPDLMGLKATNVHRVIGSNPCAEENGGCSHLCLYRPQGLRCACPIGFELISDMKTCIVPEAFLLFSRRADIRRISLETNNNNVAIPLTGVKEASALDFDVTDNRIYWTDISLKTISRAFMNGSALEHVVEFGLDYPEGMAVDWLGKNLYWADTGTNRIEVSKLDGQHRQVLVWKDLDSPRALALDPAEGFMYWTEWGGKPKIDRAAMDGSERTTLVPNVGRANGLTIDYAKRRLYWTDLDTNLIESSNMLGLNREVIADDLPHPFGLTQYQDYIYWTDWSRRSIERANKTSGQNRTIIQGHLDYVMDILVFHSSRQSGWNECASSNGHCSHLCLAVPVGGFVCGCPAHYSLNADNRTCSAPTTFLLFSQKSAINRMVIDEQQSPDIILPIHSLRNVRAIDYDPLDKQLYWIDSRQNMIRKAQEDGSQGFTVVVSSVPSQNLEIQPYDLSIDIYSRYIYWTCEATNVINVTRLDGRSVGVVLKGEQDRPRAVVVNPEKGYMYFTNLQERSPKIERAALDGTEREVLFFSGLSKPIALALDSRLGKLFWADSDLRRIESSDLSGANRIVLEDSNILQPVGLTVFENWLYWIDKQQQMIEKIDMTGREGRTKVQARIAQLSDIHAVKELNLQEYRQHPCAQDNGGCSHICLVKGDGTTRCSCPMHLVLLQDELSCGEPPTCSPQQFTCFTGEIDCIPVAWRCDGFTECEDHSDELNCPVCSESQFQCASGQCIDGALRCNGDANCQDKSDEKNCEVLCLIDQFRCANGQCIGKHKKCDHNVDCSDKSDELDCYPTEEPAPQATNTVGSVIGVIVTIFVSGTVYFICQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAPSRRMTSVATAKGYTSDLNYDSEPVPPPPTPRSQYLSAEENYESCPPSPYTERSYSHHLYPPPPSPCTDSS

SEQ ID NO: 3 - RNA, 19 nt, Artificial Sequence
  siRNA specific for human axin
ugccaagaaggcugagucg

SEQ ID NO: 4 - protein, 41 aa, Homo sapiens
  MOD_RES (4)..(4): PHOSPHORYLATION
THRSYSYRPYSYRHFAPPTTPCSTDVCDSDYAPSRRMTSVA

SEQ ID NO: 5 - protein, 4 aa, Artificial Sequence
  Consensus PKA phosphorylation site
RRXX

SEQ ID NO: 6 - protein, 5 aa, Artificial Sequence
  Consensus sequence for GSK3 phosphorylation site
SXXXX

SEQ ID NO: 7 - protein, 10 aa, Homo sapiens
  MISC_FEATURE (1)..(10): PKA primed GSK3 site
APSRRMTSVA

SEQ ID NO: 8 - protein, 4 aa, Artificial sequence
  Consensus PKA phorphorylation site in LRP6
RRKX

SEQ ID NO: 9 - protein, 4 aa, Artificial sequence
  Consensus PKA phosphorylation site in LRP6
RXXX

SEQ ID NO: 10 - protein, 3 aa, Artificial sequence
  Consensus PKA phosphorylation site in LRP6
RXX

(19) United States
(12) Patent Application Publication
(10) Pub. No.: US 2012/0294871 A1
(43) Pub. Date: Nov. 22, 2012

(54) MODULATION OF GPCR-MEDIATED CAMP PRODUCTION THROUGH LRP6 AND ITS THERAPEUTIC APPLICATION

(75) Inventors: Xu Cao, Baltimore, MD (US); Mei Wan, Ellicott City, MD (US)

(73) Assignee: THE JOHNS HOPKINS UNIVERSITY, Baltimore, MD (US)

(21) Appl. No.: 13/474,200

(22) Filed: May 17, 2012

Related U.S. Application Data
(60) Provisional application No. 61/487,160, filed on May 17, 2011.
(60) Provisional application No. 61/525,023, filed on Aug. 18, 2011.

(51) Int. Cl. G01N 33/566; C12Q 1/48; A61K 31/713; C12Q 1/68; A61K 38/02; A61K 31/7088; A61K 39/395; C12Q 1/02

(52) U.S. Cl. 424/172.1; 435/7.21; 435/7.23; 435/15; 435/29; 435/6.13; 514/1.1; 514/44 R; 514/44 A

(57) ABSTRACT

The present invention relates to low-density lipoprotein receptor related protein 6 (LRP6). More specifically, the present invention relates to targeting of LRP6 to modulate Gαₛ activity. In one embodiment, a method of screening for antagonists of IRP6-Gαₛ interaction comprises the steps of (a) contacting an agent that binds LRP6 with a cell; and (b) measuring the level of cAMP production in the cell in response to one or more GPCR ligands, wherein an agent that decreases cAMP production as compared to cAMP production in the cell not contacted with the agent identifies the agent as an antagonist of LRP6-Gαₛ interaction.

### CROSS-REFERENCE TO RELATED APPLICATIONS

This application claims the benefit of U.S. Provisional Application No. 61/525,023, filed Aug. 18, 2011, and U.S. Provisional Application No. 61/487,160, filed May 17, 2011; both of which are incorporated herein by reference in their entireties.

### STATEMENT OF GOVERNMENTAL INTEREST

This invention was made with U.S. government support under grant no. DK083350 and grant no. DK057501. The U.S. government has certain rights in the invention.

### FIELD OF THE INVENTION

The present invention relates to low-density lipoprotein receptor-related protein 6 (LRP6). More specifically, the present invention relates to targeting of LRP6 to modulate Gαₛ activity.

### INCORPORATION-BY-REFERENCE OF MATERIAL SUBMITTED ELECTRONICALLY

This application contains a sequence listing. It has been submitted electronically via EFS-Web as an ASCII text file entitled “P11565-03_ST25.txt.” The sequence listing is 31,093 bytes in size, and was created on May 16, 2012. It is hereby incorporated by reference in its entirety.

### BACKGROUND OF THE INVENTION

Cyclic adenosine monophosphate (cAMP) acts as a second messenger in prokaryotes and eukaryotes. The concentration of cytosolic cAMP is increased by an order of magnitude within seconds of the activation of heterotrimeric guanine nucleotide-binding protein (G protein)-coupled receptors (GPCRs), which typically have seven plasma membrane-spanning domains (1-3). This large family of receptors mediates many critical signaling events, including the signaling of parathyroid hormone (PTH) in bone and kidney (4-6), adrenaline in heart and muscle (7, 8), glucagon in liver and fat (9, 10), vasopressin in kidney (11, 12), adrenocorticotropic hormone in the adrenal cortex (13, 14), and luteinizing hormone in the ovary (15, 16). The rapid synthesis of cAMP is achieved by the transmembrane enzyme adenylyl cyclase (AC), which is activated directly by the αₛ subunit of the G protein that is associated with the GPCR.

The currently accepted paradigm for the production of cAMP is that the inactive form of Gαₛ is bound to guanosine diphosphate (GDP) at its guanine nucleotide-binding pocket and that Gαₛ-GDP combines with the βγ subunits of the G protein to form a heterotrimer with an inactive configuration at the cell membrane (1-3). The heterotrimer is thought to be attached to the cell membrane by hydrophobic interactions through lipid modifications of the G proteins, such as palmitoylation of Gαₛ (17-19) and isoprenylation of Gγ (20, 21). The binding of ligand to a GPCR alters the conformation of the associated Gαₛ, promoting the release of GDP and the binding of guanosine triphosphate (GTP), as well as the depalmitoylation and disassociation of Gαₛ from the βγ dimer. Gαₛ then associates with and activates AC, which results in the synthesis of cAMP (22).

Low-density lipoprotein receptor-related protein 6 (LRP6) belongs to the low-density lipoprotein receptor (LDLR) family (23, 24) and is widely abundant in human and mouse tissues. LRP6 has a large extracellular domain that contains 1372 amino acid residues anchored to the plasma membrane through a transmembrane domain that is followed by a relatively short cytoplasmic domain of 207 amino acid residues. LRP6 was initially characterized as a co-receptor that stabilizes b-catenin in the Wnt signaling pathway (25, 26), and its signaling is regulated by a large number of extracellular proteins, including members of the Dickkopf (Dkk) family (23, 27, 28) and sclerostin (29-31), in the absence of Wnts, b-catenin is found in a large cytoplasmic complex that consists of other proteins that promote its inactivation by phosphorylation and proteasomal degradation. In the presence of Wnts, Frizzled proteins, which are receptors for Wnts and share the basic structural organization of GPCRs, form complexes with their co-receptor LRP6. Phosphorylation of LRP6 at PPPS/TP motifs is then triggered, which is followed by the recruitment of axin to the plasma membrane (23), leading to inhibition of the phosphorylation and degradation of b-catenin. Stabilized b-catenin protein accumulates in the nucleus and forms complexes with the T cell factor-lymphoid enhancer factor (TCF-LEF) family of DNA binding transcription factors to enhance the expression of target genes to regulate cellular activities.

GPCRs other than Frizzled proteins, such as prostaglandin F2 receptor FPB (32), M1 acetylcholine muscarinic receptor (33), lysophosphatidic acid receptors (34), the prostaglandin E2 receptor EP2 (35), gonadotrophin releasing hormone receptor (36), and PTH receptor 1 (PTH1R) (37), also activate b-catenin signaling. In particular, the direct association of the active form of Gαₛ with the scaffolding protein axin regulates EP2-induced b-catenin signaling (35). Free Gβγ recruits glycogen synthase kinase 3 (GSK-3) to the plasma membrane to phosphorylate LRP6 at its PPPS/TP site, providing further evidence for the direct involvement of G proteins in the GPCR-stimulated stabilization of b-catenin (38). LRP6 likely acts as a common receptor in different GPCR signaling pathways mediated by the activation of G proteins because LRP6 forms complexes with various GPCRs in response to ligand stimulation (37).

### SUMMARY OF THE INVENTION

The present invention is based, at least in part, on the discovery that LRP6 was involved in Gαₛ mediated cAMP signaling. The present inventors found that knockdown of LRP6 inhibited the production of cAMP in response to different GPCR ligands. Upon ligand stimulation, LRP6 was phosphorylated by cAMP-dependent protein kinase (PKA), leading to its association with the Gαₛβγ heterotrimer, which then accumulated at the plasma membrane to enable its coupling with GPCRs to trigger cAMP production.

The discovery that the binding of Gαₛ to LRP6 is required to establish a functional GPCR-Gαₛ-AC signaling pathway for the production of cAMP provides an additional regulatory component to the current GPCR-cAMP paradigm. The central role of LRP6 in the Gαₛ-associated production of cAMP in response to GPCR stimulation in mammalian cells has a number of implications. Regulation of the production of cAMP by LRP6 may represent a previously uncharacterized control point that modifies the effects of ligand binding by Gαₛ-associated GPCRs on transcription and other cell functions. This mechanism could potentially integrate hormonal and other Gαₛ-coupled GPCR signals with other extracellular signals. Modulation of cAMP production through agents that target intracellular AC activity is targeted clinically in the treatment of many different diseases. Targeting of LRP6 by its agonists or antagonists to modulate Gαₛ activity could provide another potential therapeutic approach.

Thus, in one aspect, the present invention is useful in screening for modulators of LRP6. In one embodiment, a method of screening for antagonists of LRP6-Gαₛ interaction comprises the steps of (a) contacting an agent that binds LRP6 with a cell; and (b) measuring the level of cAMP production in the cell in response to one or more GPCR ligands, wherein an agent that decreases cAMP production as compared to cAMP production in the cell not contacted with the agent identifies the agent as an antagonist of LRP6-Gαₛ interaction. In a specific embodiment, Gαₛ is complexed with Gβγ in a heterotrimer. In another embodiment, the GPCR ligand is PTH(1-34) and the cell is an osteosarcoma cell. In an alternative embodiment, the GPCR ligand is isoproterenol and the cell is an osteoprogenitor cell. In other embodiments, the GPCR ligand is adenosine and the cell is a bronchial smooth muscle cell. In a further embodiment, the GPCR ligand is glucagon and the cell is an embryonic kidney cell. Alternatively, the GPCR ligand is isoproterenol and the cell is an embryo fibroblast. In certain embodiments, the method further comprises contacting the cell with forskolin. In particular embodiments of the present invention, the agent is a small molecule, an antibody, polypeptide, a polynucleotide, an aptamer, or an siRNA. In specific embodiments, a method for treating an LRP6-mediated disease can comprise the step of administering to a patient an effective amount of an antagonist identified using a method described herein.

In another aspect, the present invention provides methods useful for identifying LRP6 modulators. In one embodiment, a method for identifying an LRP6 modulator comprises the step of performing a kinase assay using LRP6 and cAMP-dependent protein kinase (PKA) in vitro in the presence and absence of a test agent, wherein an agent that inhibits or reduces the phosphorylation of LRP6 by PKA, is identified as an LRP6 modulator. In particular embodiments, the agent is a small molecule, an antibody, polypeptide, a polynucleotide, an aptamer, or an siRNA. In specific embodiments, a method for treating an LRP6-mediated disease comprises the step of administering to a patient an effective amount of an LRP6 modulator identified using a method described herein.

In another embodiment, the present invention provides a method of screening for LRP6 modulators comprising the steps of (a) contacting a cell that expresses LRP6 with an agent; (b) assaying LRP6 activity; and (c) comparing the assayed LRP6 activity to LRP6 activity in cell that has not been contacted with the agent, wherein a difference in the compared LRP6 activities identifies the agent as an LRP6 modulator. LRP6 activity can be determined, for example, by measuring the level of LRP6 mRNA, measuring the level of β-catenin, or measuring the level of cAMP production. In particular embodiments, the agent is a small molecule, an antibody, polypeptide, a polynucleotide, an aptamer, or an siRNA. In specific embodiments, a method for treating an LRP6-mediated disease can comprise the step of administering to a patient an effective amount of an antagonist identified using a method described herein.

In one embodiment, a method of screening for therapeutic agents useful in the treatment of LRP6-mediated diseases comprises the steps of (a) contacting a test agent with an LRP6 polypeptide; and (b) detecting the binding of the test agent to the LRP6 polypeptide. In other embodiments, the method can further comprise (c) contacting the test agent with a cell derived from a patient suffering from an LRP6-mediated disease; and (d) determining the effect of the test agent on the cell In particular embodiments, the agent is a small molecule, an antibody, polypeptide, a polynucleotide, an aptamer, or an siRNA. In specific embodiments, a method for treating LRP6-mediated disease can comprise the step of administering to a patient an effective amount of an antagonist identified using a method described herein.

### BRIEF DESCRIPTION OF THE FIGURES

FIG. 1. LRP6 is required for PTH-induced production of cAMP. (A) LRP6-specific siRNA inhibited PTH-induced cAMP accumulation in UMR-106 cells. Cells were transfected with siRNAs specific for GFP, LRP5, or LRP6 or with siRNAs against LRP5 and LRP6. Cells were treated with PTH(1-34) (50 nM) for 30 min and collected and the amount of accumulated [3H]cAMP was measured. Data are the means±SD from three experiments. #P<0.05, *P<0.001 relative to the control cells treated with siRNA against GFP. (B) LRP6-specific siRNA inhibited PTH-induced cAMP accumulation in C2C12 cells. Cells were transfected, treated, and analyzed as described for (A). Data are the means±SD from three experiments. *P<0.001 relative to control cells treated with siRNA against GFP. (C) Accumulation of cAMP in response to PTH was abolished in LRP6-deficient primary calvarial preosteoblasts. Calvarial preosteoblasts were isolated from lrp6-floxed or lrp5−/−; lrp6-floxed newborn mice, infected with adenovirus expressing GFP or Cre, and treated with PTH(1-34) (50 nM) for 30 min. Cells were collected, and the amount of accumulated [3H]cAMP was measured. Data are the means±SD from three experiments, *P<0.001 relative to wild-type (WT) cells. (D) LRP6-specific siRNA attenuated the PTH-induced phosphorylation of CREB in UMR-106 cells. Cells were transfected and treated as described for (A). Cells were then collected and subjected to Western blotting analysis with antibodies against pCREB and total CREB. Data are representative of three experiments. (E) Representative emission ratio time courses (from six experiments) of ICUE3 stimulated with PTH(1-34) (50 nM) or forskolin (50 nM). (F) Representative emission ratio time courses (from six experiments) of ICUE3 stimulated with PTH(1-3) (50 nM) in control, LRP5 knockdown, or LRP6 knockdown cells.

FIG. 2. LRP6 is required for receptor induced, Gαₛ-coupled cAMP production. (A) LRP6-specific siRNA abolished isoproterenol-induced cAMP accumulation in C2C12 cells. Cells were transfected with siRNAs against GFP, LRP5, or LRP6 or with siRNAs specific for LRP5 and LRP6. Cells were treated with isoproterenol (Iso, 10 nM) for 20 min., and the amount of accumulated [3H]cAMP was measured. Data are the means±SD from three experiments. *P<0.001 relative to control cells treated with siRNA against GFP. (B) LRP6-specific siRNA abolished adenosine-induced cAMP accumulation in bronchial smooth muscle cells. Cells were transfected as described for (A) and treated with adenosine (10 nM) for 30 min, and the amount of accumulated [3H]cAMP was measured. Data are the means±SD from three experiments. *P<0.005 relative to control cells treated with siRNA against GFP. (C) LRP6-specific siRNA abolished glucagon-induced cAMP accumulation in HEK 293 cells. Cells were transfected with plasmid encoding the glucagon receptor and with siRNAs specific for GFP, LRP5, or LRP6 or with siRNA against the common region of LRP5 and LRP6, treated with glucagon (50 nM) for 30 min, and the amount of accumulated [3H]cAMP was measured. Data are the means±SD from three experiments. *P<0.005 relative to control cells treated with siRNA specific for GFP. (D) The accumulation of cAMP in LRP6-deficient MEFs in response to forskolin was less affected than that induced by isoproterenol. MEFs were isolated from lrp6-floxed or lrp5−/−; lrp6-floxed newborn mice, infected with adenovirus expressing GFP or Cre, and treated with isoproterenol (Iso, 10 nM) or forskolin (Fors, 50 mM) for 30 min. Cells were collected, and the amount of accumulated [3H]cAMP was measured. Data are the means±SD from three experiments. #P<0.05, *P<0.001 relative to WT. (E) Representative emission ratio time courses (from six experiments) of ICUE3 stimulated with isoproterenol (10 nM) or forskolin (50 mM) in control and LRP6 knockdown cells.

FIG. 3. PTH promotes the association of LRP6 and Gαₛ. (A) Gαₛ interacts with the cytoplasmic domain of LRP5 (LRP5C) and LRP6 (LRP6C). GST, GST-LRP5C, or GST-LRP6C was incubated with HEK 293 cell lysates. Bound Gαₛ was detected by Western blotting analysis with an antibody against Gαₛ (upper panel). Gαₛ in cell lysates was detected by Western blotting analysis with an antibody against Gαₛ (middle panel). The GST proteins were visualized by Coomassie brilliant blue staining (lower panel), (B) PTH does not affect the interaction between Gαₛ and LRP5. HEK 293 cells were transfected with plasmid encoding HA-tagged LRP5, deprived of serum for 14 hours to avoid protein phosphorylation by serum-derived hormones or growth factors, and treated with PTH(1-34) (50 nM). LRP5-associated Gαₛ was detected by Western blotting analysis of samples that were subjected to immunoprecipitation (IP) with an antibody against HA. Immunoprecipitation with a mouse preimmune antibody (Pre) was also performed as a negative control. WCL, whole-cell lysate. (C) PTH increases the extent of the interaction between Gαₛ and LRP6. HEK 293 cells were transfected with plasmid encoding VSVG-tagged LRP6, deprived of serum for 14 hours, and treated with PTH(1-34) (50 nM). LRP6-associated Gαₛ was detected by Western blotting analysis of samples immunoprecipitated with antibody against VSVG. (D) The interactions of LRP6 with Gαₛ and Gb1 are blocked by AlF₄⁻. HEK 293 cells were transfected with plasmid encoding VSVG-tagged LRP6 together with plasmids encoding FLAG-tagged Gαs, HA-tagged Gb1, and Gg2 and were lysed in lysis buffer containing AlF4− (100 mM AlCl3, 10 mM NaF). LRP6-associated Gαₛ and Gβγ were detected by Western blotting analysis of samples immunoprecipitated with antibody against VSVG. (E) LRP6 does not bind to Gαₒ or Gαᵢ₂. HEK 293 cells were transfected with plasmid encoding VSVG-tagged LRP6. LRP6-associated Gαₛ, Gαₒ, and Gαᵢ₂ were detected by Western blotting analysis of samples immunoprecipitated with antibodies against Gαₛ, Gαₒ, and Gαᵢ₂. Immunoprecipitation with a mouse preimmune antibody (Pre) was also performed as a negative control. (F) The PTH-induced interaction between Gαₛ and LRP6 was not affected by knockdown of axin. HEK 293 cells were transfected with scrambled control siRNA or siRNA specific for axin together with the indicated plasmids and treated with PTH(1-34) (50 nM). LRP6-associated Gαₛ was detected by Western blotting analysis of samples immunoprecipitated with antibody against VSVG.

FIG. 4. PTH induces LRP6 aggregation and Gαₛβγ accumulation in UMR-106 cells, as analyzed by sucrose gradient sedimentation. UMR-106 cells were transfected with siRNAs specific fir GFP or LRP6, and were treated with PTH(1-34) (50 nM) for the indicated time periods. Cell lysates were subjected to sucrose density-gradient ultracentrifugation, and fractions were analyzed by Western blotting with antibodies against LRP6. Gαₛ, Gβ₁, and Gγ₂. Data are representative of three experiments.

FIG. 5. Knockdown of LRP6 impairs the targeting of Gαₛ to the plasma membrane. (A) Localization of Gαₛ and Gβ₁ to the plasma membrane is impaired by knockdown of LRP6. Stable HEK 293 cell lines expressing GFP-Gαₛ, GFP-Gβ₁, or CFPPTH1R were transfected with siRNAs specific for LRP5 or LRP6 or with siRNA containing a random sequence (Ctrl). Green fluorescence was visualized by fluorescence microscopy. (B) Cells from (A) in which Gαₛ, Gβ₁, or PTH1R was localized primarily at the plasma membrane or in the cytosol were quantified. For each treatment, 100 cells on three different slides were analyzed. The number of cells in which fluorescent protein was localized to either the plasma membrane (black bars) or the cytosol (white bars) divided by the total number of cells was calculated and expressed as a percentage±SD. *P<0.05 relative to siRNA control cells. (C) Localization of Gαₛ to the plasma membrane is impaired by deletion of LRP6 in osteoblasts from mouse femur. Immunohistochemical analysis of the amount of Gαₛ in sections of femurs from 2-month-old male lrp6-floxed (WI) and lpr6-floxed; OC-Cre (KO) mice. Photos are representative of tissue sections stained for Gαₛ and counterstained with methyl green. Gαₛ-containing osteoblasts were stained brown. Three random high-power fields per specimen and a total of six specimens in each group were analyzed. The image shown is from one of these specimens. (D) Osteoblasts from (C) in which Gαₛ was localized to the plasma membrane or to the cytosol were counted in a blinded fashion from three random high-power fields per specimen in a 2-mm square, 1 mm distal to the lowest point of the growth plate in the secondary spongiosa; a total of six specimens in each group were used. The osteoblasts in which Gαₛ was localized either to the plasma membrane (black bars) or to the cytosol (white bars) are presented as a percentage of the total number of osteoblasts. *P<0.05 relative to WT samples. (E and F) The interaction between Gαₛ and PTH1R is attenuated by knockdown of LRP6. HEK 293 cells were transfected with siRNA specific for GFP or LRP6. Reciprocal immunoprecipitations were performed to identify the interaction between PTH1R and Gαₛ. Data are representative of three experiments.

FIG. 6. Phosphorylation of LRP6 by PKA, enhances the binding of LRP6 to Gαₛ. (A) PKA directly phosphorylates the intracellular domain of LRP6, as determined by in vitro kinase assay. The consensus PKA phosphorylation sites in the intracellular domain of LRP6 are shown at the top. GST, GST-LRP5C, and GST-LRP6C proteins were pulled down by glutathione beads. The bead-bound proteins were incubated with the recombinant catalytic subunit of PKA and [g-32P]ATP, and the protein-associated radiolabel was visualized by PhosphorImager analysis (Autoradiography). The GST proteins were analyzed by SDS-PAGE and visualized by Coomassie Brilliant Blue staining. Data are representative of three experiments. (B) Mutation of Thr1548 of LRP6 abolishes the phosphorylation of LRP6C by PKA. WT or various point-mutated GST-6C proteins were pulled down by glutathione beads. The bead-bound proteins were incubated with the catalytic subunit of PKA and [γ-32^{P}]ATP, and the protein-associated radiolabel was analyzed by SDS-PAGE and visualized by PhosphorImager analysis (Autoradiography). The GST proteins were visualized by Coomassie Brilliant Blue staining. Data are representative of three experiments. (C) Phosphorylation of LRP6C by PKA primes its phosphorylation by GSK-3. The consensus PKA and GSK-3 phosphorylation sites are shown at the top. GST-6C proteins were pulled down by glutathione beads. The bead-bound proteins were incubated with the catalytic subunit of PKA and cold ATP, washed, and then incubated with recombinant GSK-3 and [γ-32^{P}]ATP. The protein-associated radiolabel was analyzed by SDSPAGE and visualized by PhosphorImager analysis (Autoradiography). The GST proteins were visualized by Coomassie brilliant blue staining. Data are representative of three experiments. (D) Mutation of the PKA or GSK-3 sites of LRP6 abolishes the sequential phosphorylation of LRP6C by PKA and GSK-3. WT (WT-6C) or the point-mutated GST-6C proteins, 6C^{mPKA} (Thr¹⁵⁴⁸→Ala) and 6C^{mGSK3} (Ser¹⁵⁴⁴→Ala), were pulled down by glutathione beads. The bead-bound proteins were then treated and analyzed as described for (A). The GST proteins were visualized by Coomassie Brilliant Blue staining. Data are representative of three experiments. Abbreviations for the amino acids are as follows: A, Ala; C, Cys; D, Asp; F, Phe; H, His; M, Met; P, Pro; R, Arg; S, Ser; T, Thr; V, Val; and Y, Tyr.

FIG. 7. Phosphorylation of LRP6 by PKA and GSK-3 promotes the binding of Gαₛ to LRP6. (A and B) Mutation of the PKA site of LRP6 inhibits its binding to Gαₛ in vitro. GST -fused WT-6C or 6C^{mPKA} proteins were pulled down by glutathione beads. The bead-bound proteins were incubated with the catalytic subunit of PKA and ATP, washed, and then incubated with recombinant GSK-3 and ATP. The protein-associated beads were incubated with HEK 293 cell lysates. Bound Gαₛ was detected by Western blotting analysis with an antibody against Gαₛ (A, upper panel). The amounts of Gαₛ in the cell lysates and GST proteins were detected by Western blot analysis with an antibody against Gαₛ or GST (A, input panels). The intensities of the bands were quantitated by phosphorimaging and normalized to the density of the input amount of Gαₛ (B). (C) Mutation of the PKA site of LRP6 inhibits its binding to Gαₛ induced by PTH in cells. HEK 293 cells were transfected with plasmid encoding VSVG-tagged WT-LRP6 or LRP6^{mPKA}. Cells were deprived of serum for 14 hours and treated with PTH(1-34) (50 nM). Gαₛ-associated LRP6 was detected by Western blotting analysis of samples immunoprecipitated with antibody against Gαₛ. Data are representative of three experiments. (D) Mutation of the PKA site of LRP6 impairs the localization of Gαₛ to the plasma membrane. Stable HEK 293 cell lines expressing GFP-Gαₛ were transfected with empty vector (Control) or plasmids encoding VSVG-tagged WT-LRP6, LRP6^{mPKA}, or LRP6^{mGSK3}. Green fluorescence was visualized by fluorescence microscopy. Data are representative of three experiments. (E) Cells from (D) in which localization of Gαₛ was at the plasma membrane or in the cytosol were counted. For each treatment, 100 cells in three different slides were analyzed. The number of cells within which fluorescent protein was localized to the plasma membrane or the cytosol divided by the total number of cells was calculated and expressed as a percentage±SD. *P<0.05 relative to the empty vector control. (F) Accumulation of cAMP in MEFs in response to isoproterenol was reduced by mutation of the PKA or GSK-3 phosphorylation sites in LRP6. MEFs were isolated from E13.5 (embryonic day 13.5) mice and transfected with empty vector control or with plasmid encoding VSVG-tagged WT-LRP6, LRP6^{mPKA}, LRP6^{mGSK3} and treated with isoproterenol (10 nM) for 20 min. Cells were collected and the amount of accumulated [3H]cAMP was measured. *P<0.05, #P<0.005 relative to the cells containing empty vector control. (G) Representative emission ratio time courses (from eight experiments) of ICUE3 stimulated with isoproterenol (Iso, 10 nM) in cells containing WT LRP6 (WT-LRP6) or LRP6 with a mutation in the PKA phosphorylation site (LRP6-mPKA).

FIG. 8. Schematic model of the involvement of LRP6 in Gαs-coupled receptor signaling. (A) We propose that LRP6 binds to the inactive Gαₛβγ heterotrimer on the plasma membrane in the absence of GPCR ligands. (B) Ligands induce the aggregation of LRP6 and cause the accumulation of Gαₛβγ on the plasma member to set up a functional GPCR-Gαₛ-AC complex for cAMP generation and PKA activation. Activated PKA, in turn, phosphorylates LRP6 and accelerates the binding of G protein complexes to LRP6 to enable enhanced production of cAMP.

FIG. 9. Mass spectrometric identification of a PKA phosphorylation site in LRP6. (A) GST-6C protein was incubated with a recombinant catalytic subunit of PKA and ATP. Phosphorylated GST-6C protein was subjected to SDS-PAGE followed by staining with Coomassie brilliant blue. Lane 1, protein molecular mass marker; Lane 2, GST-6C alone; Lane 3, aliquot of the reaction mixture including GST-6C, PKA, and ATP; Lane 4, PKA alone. The upper band of lane 3 was collected for mass spectrometry analysis. (B) Mass spectrometry analysis showed a peak at m/z 530.791 (arrow) different from the mass of the peptide. Data are representative data of three experiments.

FIG. 10. Identification of the Gαₛ interaction domain in LRP6. (A) WT and mutant constructs used in these experiments. (B) HEK 293 cells were transfected with plasmids encoding VSVG-tagged WT LRP6 (WT) or a series of deletion constructs and were treated with PTH(1-34) (50 nM). Gαₛ-associated LRP6 was detected by Western blotting analysis with an antibody against VSVG. LRP6 and Gαₛ proteins in cell lysates were detected by Western blotting analysis with antibodies against VSVG and Gαₛ. WCL, whole-cell lysates. (C) The intensities of the bands in the Western blots were quantitated by phosphorimaging and normalized to the density of the anti-Gαₛ blots, that is, the ratio of the band density in the first row to the band density in the second row. Data are representative data of three experiments.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

### I. Definitions

As used herein, the term “modulate” indicates the ability to control or influence directly or indirectly, and by way of non-limiting examples, can alternatively mean inhibit or stimulate, agonize or antagonize, hinder or promote, and strengthen or weaken. Thus, the term “LRP6 modulator” refers to an agent that modulates LRP6. Modulators may be organic or inorganic, small to large molecular weight individual compounds, mixtures and combinatorial libraries of inhibitors, agonists, antagonists, and biopolymers such as peptides, nucleic acids, or oligonucleotides. A modulator may be a natural product or a naturally-occurring small molecule organic compound. In particular, a modulator may be a carbohydrate; monosaccharide; oligosaccharide; polysaccharide; amino acid; peptide; oligopeptide; polypeptide; protein; receptor; nucleic acid; nucleoside; nucleotide; oligonucleotide; polynucleotide including DNA and DNA fragments, RNA and RNA fragments and the like; lipid; retinoid; steroid; glycopeptides; glycoprotein; proteoglycan and the like; and synthetic analogues or derivatives thereof, including peptidomimetics, small molecule organic compounds and the like, and mixtures thereof. A modulator identified according to the invention is preferably useful in the treatment of a disease disclosed herein.

As used herein, an “antagonist” is a type of modulator and the term refers to an agent that binds a target (e.g., a protein) and can inhibit a one or more functions of the target. For example, an antagonist of a protein can bind the protein and inhibit the binding of a natural or cognate ligand to the protein and/or inhibit signal transduction mediated through the protein.

An “agonist” is a type of modulator and refers to an agent that binds a target and can activate one or more functions of the target. For example, an agonist of a protein can bind the protein and activate the protein in the absence of its natural or cognate ligand.

As used herein, the term “antibody” is used in reference to any immunoglobulin molecule that reacts with a specific antigen. It is intended that the term encompass any immunoglobulin (e.g., IgG, IgM, IgA, IgE, IgD, etc.) obtained from any source (e.g., humans, rodents, non-human primates, caprines, bovines, equines, ovines, etc.). Specific types/examples of antibodies include polyclonal, monoclonal, humanized, chimeric, human, or otherwise-human-suitable antibodies. “Antibodies” also includes any fragment or derivative of any of the herein described antibodies. In specific embodiments, antibodies may be raised against LRP6 and used as LRP6 modulators.

The terms “specifically binds to,” “specific for,” and related grammatical variants refer to that binding which occurs between such paired species as antibody/antigen, enzyme/substrate, receptor/agonist, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody typically binds to a single epitope and to no other epitope within the family of proteins. In some embodiments, specific binding between an antigen and an antibody will have a binding affinity of at least 10⁻⁶ M. In other embodiments, the antigen and antibody will bind with affinities of at least 10⁻⁷ M, 10⁻⁸ M to 10⁻⁹ M, 10⁻¹⁰ M, 10⁻¹¹ M, or 10⁻¹² M.

Optional” or “optionally” means that the subsequently described event or circumstance can or cannot occur, and that the description includes instances where the event or circumstance occurs and instances where it does not.

“Bone development” generally refers to any process involved in the change of bone over time, including, for example, normal development, changes that occur during disease states, and changes that occur during aging. This may refer to structural changes in and dynamic rate changes such as growth rates, resorption rates, bone repair rates, and etc. “Bone development disorder,” as further defined below, particularly refers to any disorders in bone development including, for example, changes that occur during disease states and changes that occur during aging. Bone development may be progressive or cyclical in nature. Aspects of bone that may change during development include, for example, mineralization, formation of specific anatomical features, and relative or absolute numbers of various cell types.

“Bone modulation” or “modulation of bone formation” refers to the ability to affect any of the physiological processes involved in bone remodeling, as will be appreciated by one skilled in the art, including, for example, bone resorption and appositional bone growth, by, inter alia, osteoclastic and osteoblastic activity, and may comprise some or all of bone formation and development as used herein.

Bone is a dynamic tissue that is continually adapting and renewing itself through the removal of old or unnecessary bone by osteoblastic and the rebuilding of new bone by osteoblasts. The nature of the coupling between these processes is responsible both for the modeling of bone during growth as well as the maintenance of adult skeletal integrity through remodeling and repair to meet the everyday needs of mechanical usage. There are a number of diseases of bone that result from an uncoupling of the balance between bone resorption and formation. With aging there is a gradual “physiologic” imbalance in bone turnover, which is particularly exacerbated in women due to menopausal loss of estrogen support, that leads to a progressive loss of bone. The reduction in bone mass and deterioration in bone architecture results in an increase in bone fragility and susceptibility to spontaneous fractures. For every 10 percent of bone that is lost the risk of fracture doubles. Individuals with bone mineral density (BMD) in the spine or proximal femur about 2.5 or more standard deviations below normal peak bone mass are classified as osteoporotic. However, osteopenic individuals with BMD between about 1 and about 2.5 standard deviations below the norm are clearly at risk of suffering bone loss related disorders.

Bone modulation may be assessed by measuring parameters such as bone mineral density (BMD) and bone mineral content (BMC) by pDXA X-ray methods, bone size, thickness or volume as measured by X-ray, bone formation rates as measured for example by calcien labeling, total, trabecular, and mid-shaft density as measured by pQCT and/or mCT methods, connectivity and other histological parameters as measured by mCT methods, mechanical bending and compressive strengths as preferably measured in femur and vertebrae respectively. Due to the nature of these measurements, each may be more or less appropriate for a given situation as the skilled practitioner will appreciate. Furthermore, parameters and methodologies such as a clinical history of freedom from fracture, bone shape, bone morphology, connectivity, normal histology, fracture repair rates, and other bone quality parameters are known and used in the art. In certain embodiments, bone quality may be assessed by the compressive strength of vertebra, when such a measurement is appropriate. Bone modulation may also be assessed by rates of change in the various parameters. In certain instances, bone modulation is assessed at more than one age.

“Normal bone density” refers to a bone density within two standard deviations of a Z score of 0 in the context of the HBM linkage study. In a general context, the range of normal bone density parameters is determined by routine statistical methods. A normal parameter is within about 1 or 2 standard deviations of the age and sex normalized parameter, preferably about 2 standard deviations. A statistical measure of meaningfulness is the P value which can represent the likelihood that the associated measurement is significantly different from the mean. Significant P values are P<0.05, 0.01, 0.005, and 0.001, preferably at least P<0.01.

As used herein, a “subject” or “patient” means an individual and can include domesticated animals, (e.g., cats, dogs, etc.); livestock (e.g., cattle, horses, pigs, sheep, goats, etc.), laboratory animals (e.g., mouse, rabbit, rat, guinea pig, etc.) and birds. In one aspect, the subject is a mammal such as a primate or a human. In particular, the term also includes mammals diagnosed with an LRP6 mediated disease, disorder or condition.

The terms “subject” and “patient” are used interchangeably here, and are intended to include organisms, e.g., eukaryotes, which are suffering from or afflicted with a disease, disorder or condition associated with LRP6. Examples of subjects or patients include mammals, e.g., humans, dogs, cows, horses, pigs, sheep, goats, cats, mice, rabbits, rats, and transgenic non-human animals. In certain embodiments, the subject or patient is a human, e,g., a human suffering from, at risk of suffering from, or potentially capable of suffering from cancer (e.g., colon cancer) and other proliferative diseases, osteoporosis, and schizophrenia, and other diseases or conditions described herein (e.g., an LRP6-related disease, disorder or condition).

As used herein, the term “effective,” means adequate to accomplish a desired, expected, or intended result. More particularly, a “therapeutically effective amount” as provided herein refers to an amount of an LRP6 modulator of the present invention, either alone or in combination with another therapeutic agent, necessary to provide the desired therapeutic effect, e.g., an amount that is effective to prevent, alleviate, or ameliorate symptoms of disease or prolong the survival of the subject being treated. In a specific embodiment, the term “therapeutically effective amount” as provided herein refers to an amount of an LRP6 modulator, necessary to provide the desired therapeutic effect, e.g., an amount that is effective to prevent, alleviate, or ameliorate symptoms of disease or prolong the survival of the subject being treated. As would be appreciated by one of ordinary skill in the art, the exact amount required will vary from subject to subject, depending on age, general condition of the subject, the severity of the condition being treated, the particular compound and/or composition administered, and the like. An appropriate “therapeutically effective amount” in any individual case can be determined by one of ordinary skill in the art by reference to the pertinent texts and literature and/or by using routine experimentation.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease.

The terms “LRP6-related disease, disorder or condition” or “LRP6-mediated disease, disorder or condition,” and the like mean diseases, disorders or conditions associated with aberrant LRP6 signaling, including but not limited to cancers (e.g., colorectal carcinomas (CRCs), melanoma, breast, liver, lung, and gastric cancer; other, non-oncogenic proliferative diseases, such as proliferative skin disorders (e.g., psoriasis, dermatitis); osteoporosis; osteoarthritis; fibrotic disorders; schizophrenia; vascular disease; cardiac disease; aberrant hair growth or difficulty growing hair; wound healing; regenerative needs (liver, lung, limb); and neurodegenerative diseases such as Alzheimer's disease.

any abnormal state that involves LRP6 activity. The abnormal state can be induced by environmental exposure or drug administration. Alternatively, the disease or disorder can be due to a genetic defect. LRP6-mediated diseases, disorders and conditions include, but are not limited to, bone related disorders or conditions and lipid disorders and conditions. For example, bone mass disorders/conditions/diseases, which may be mediated by LRP6 include, but are not limited, to age related loss of bone, bone fractures (e.g., hip fracture, Colle's fracture, vertebral crush fractures), chondrodystrophies, drug-induced disorders (e.g., osteoporosis due to administration of glucocorticoids or heparin and osteomalacia due to administration of aluminum hydroxide, anticonvulsants, or glutethimide), high bone turnover, hypercalcemia, hyperostosis, osteoarthritis, osteogenesis imperfecta, osteomalacia, osteomyelitis, osteoporosis, Paget's disease, and rickets.

Lipid disorders/diseases/conditions, which may be mediated by Dkk, LRP5, and/or LRP6 include but are not limited to familial lipoprotein lipase deficiency, familial apoprotein CII deficiency, familial type 3 hyperlipoproteinemia, familial hypercholesterolemia, familial hypertriglyceridemia, multiple lipoprotein-type hyperlipidemia, elevated lipid levels due to dialysis and/or diabetes, and elevated lipid levels of unknown etiologies

As used herein, the term “bone-related disorder” includes disorders in which bone mineral density (BMD) is abnormally and/or pathologically high relative to healthy subjects, and disorders in which bone mineral density (BMD) is abnormally and/or pathologically low relative to healthy subjects. Disorders characterized by high BMD include but are not limited to sclerostrosis, Van Buchem disease, bone overgrowth disorders, and Simpson-Golabi-Behtnel syndrome (SGBS). Disorders characterized by low BMD and/or bone fragility include but are not limited to primary and secondary osteoporosis, osteopenia, osteomalacia, osteroporosis-pseudoglioma syndrome (OPPG), osteogenesis imperfecta (OI), avascular necrosis (osteonecrosis), fractures and implant healing (dental implants and hip implants), and bone loss due to other disorders (e.g., associated with HIV infection, cancers, or arthritis). Other “bone-related disorders” include but are not limited to rheumatoid arthritis, osteoarthritis, fracture, arthritis, and the formation and/or presence of osteolytic lesions.

As used herein, “fibrotic disorders” means any fibrotic disorder in humans characterized by excessive fibroblast or myofibroblast proliferation and production of connective tissue matrix, including collagen, fibronectin and glycosaminoglycans (GAG). Such disorders include, but are not limited to, cutaneous keloid formation; progressive systemic sclerosis (PSS); liver cirrhosis; idiopathic and pharmacologically induced pulmonary fibrosis; chronic graft versus-host disease; scleroderma (local and systemic); Peyronie's disease; post-cystoscopic urethral stenosis; post-surgical internal adhesions; idiopathic and pharmacologically induced retroperitoneal fibrosis; and myelofibrosis.

As used herein, “fibrotic disorders” also include, but are not limited to, lung fibrotic disorders (e.g., radiation-induced fibrosis, and fibrosis associated with asthma, COPD, and sarcoidosis); liver fibrotic disorders (e.g., alcoholic, Hepatitis C-associated, and primary biliary fibrosis, as well as non-alcoholic steatosis, sclerosing cholangitis, and fibrosis resulting from schistosomiasis); kidney fibrotic disorders (e.g., diabetic nephropathy, lupus glomerulosclerosis, Alport syndrome, and chronic renal allograft rejection); cardiovascular fibrotic disorders (e,g., post myocardial infarction scarring, cardiac hypertrophy, arterial restenosis, and atherosclerosis); skin fibrotic disorders (e.g., hypertrophic scarring, burn scarring, and nephrogenic fibrosing dermatopathy); and eye fibrotic disorders (e.g., vitreoretinopathy and retroorbital fibrosis)

### II. LRP6 Polynucleotides, Polypeptides and Expression Thereof

In particular embodiments, LRP6 is human. In other embodiments, LRP6 and is non-human (e.g., primate, rodent, canine, or feline). There are a variety of sequences that are disclosed on GenBank, at www.pubmed.gov, and these sequences and others herein are incorporated by reference in their entireties as are individual subsequences or fragments contained therein. As used herein, LRP6 refers to the LRP6 co-receptor that acts synergistically with the Frizzled (Fz)-receptor family members to bind Wnt and activate downstream signaling in the Wnt signaling pathway. For example, the nucleotide and amino acid sequences or the human LRP6 can be found at GenBank Accession Nos. NM_{—}002336 (SEQ ID NO:1) and NP_{—}002327, respectively (SEQ ID NO:2). Thus provided are the nucleotide sequences of LRP6 as well as nucleotide sequences at least about 70%, 75%, 80%, 85%, 90%, 95%, 98%, 99% or more, identical to the nucleotide sequence of the aforementioned GenBank Accession Nunibers. Also provided are amino acid sequences of LRP6 as well as amino acid sequence at least about 70%, 75%, 80%, 85%, 90%, 95%, 98%, 99% or more, identical to the sequences of the aforementioned GenBank Accession Numbers.

As with all peptides, polypeptides, and proteins, including fragments thereof, it is understood that additional modifications in the amino acid sequence of the LRP6 polypeptides can occur that do not alter the nature or function of the peptides, polypeptides, or proteins. Such modifications include conservative amino acids substitutions.

The polypeptides described herein can be further modified and varied so long as the desired function is maintained. It is understood that one way to define any known modifications and derivatives or those that might arise, of the disclosed genes and proteins herein is through defining the modifications and derivatives in terms of identity to specific known sequences. Specifically disclosed are polypeptides which have at least 70, 71, 72, 73, 74, 75, 76, 77, 78, 79, 80, 81, 82, 83, 84, 85, 86, 87 88, 89, 90, 91, 92. 93, 94, 95, 96, 97, 98, and 99 percent identity to LRP6 and variants provided herein. Those of skill in the art readily understand how to determine the identity of two polypeptides. For example, the identity can be calculated after aligning the two sequences so that the identity is at its highest level.

Another way of calculating identity can be performed by published algorithms. Optimal alignment of sequences for comparison may be conducted by the local identity algorithm of Smith and Waterman, Adv. Appl. Math 2:482 (1981), by the identity alignment algorithm of Needleman and Wunsch, J. Mol. Biol. 48: 443 (1970), by the search for similarity method of Pearson and Lipman, Proc. Natl. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of identity can be obtained for nucleic acids by, for example, the algorithms disclosed in Zuker, Science 244:48-52 (1989), Jaeger et al., Proc. Natl. Acad. Sci. USA 86:7706-7710 (1989), Jaeger et al., Methods Enzymol. 183:281-306 (1989), which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity and to be disclosed herein.

Protein modifications include amino acid sequence modifications. Modifications in amino acid sequence may arise naturally as allelic variations due to genetic polymorphism) or may be produced by human intervention (e.g., by mutagenesis of cloned DNA sequences), such as induced point, deletion, insertion, and substitution mutants. These modifications can result in changes in the amino acid sequence, provide silent mutations, modify a restriction site, or provide other specific mutations. Post-translational modifications can include variations in the type or amount of carbohydrate moieties of the protein core or any fragment or derivative thereof. Amino acid sequence modifications typically fall into one or more of three classes: substitutional, insertional, or deletional modifications. Insertions include amino and/or terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. Typically, no more than about from two to six residues are deleted at any one site within the protein molecule. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e., a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional modifications are those in which at least one residue has been removed and a different residue inserted in its place.

Modifications, including the specific amino acid substitutions, are made by known methods. By way of example, modifications are made by site-specific mutagenesis of nucleotides in the DNA encoding the protein, thereby producing DNA encoding the modification, and thereafter expressing the DNA in recombinant cell culture. Techniques for making substitution mutations at predetermined sites in DNA having a known sequence are well known, for example M13 primer mutagenesis and PCR mutagenesis.

Nucleic acids that encode the polypeptide sequences, variants, and fragments thereof are disclosed. These sequences include all degenerate sequences related to a specific protein sequence, i.e., all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequences.

Isolated nucleic acid molecules can be produced by standard techniques. For example, polymerase chain reaction (PCR) techniques can be used to obtain an isolated nucleic acid containing a nucleotide sequence described herein. Various PCR methods are described, for example, in PCR Primer: A Laboratory Manual, Dieffenbach and Dveksler, eds., Cold Spring Harbor Laboratory Press, 1995. Various PCR strategies also are available by which the site-specific nucleotide sequence modifications described herein can be introduced into a template nucleic acid. Optionally, isolated nucleic acids are chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids disclosed herein also can be obtained by mutagenesis of, a naturally occurring DNA.

Nucleic acids that encode the polypeptide sequences, variants, and fragments thereof can be cloned into a vector for delivery into the cell. There are a number of compositions and methods which can be used to deliver nucleic acids to cells, either in vitro or in vivo via, for example, expression vectors. These methods and compositions can largely be broken down into two classes: viral based delivery systems and non-viral based delivery systems. Such methods are well known and readily adaptable for use with the compositions and methods described herein.

As used herein, plasmid or viral vectors transport the disclosed nucleic acids into the cell without degradation and include a promoter yielding expression of the gene in the cells into which it is delivered. Viral vectors are, for example, Adenovirus, Adeno-associated virus, Herpes virus, Vaccinia virus, Polio virus, Sindbis and other RNA viruses, including these viruses with the HIV backbone. Also preferred are any viral families which share the properties of these viruses which make them suitable for use as vectors. Retroviral vectors, in general, are described by Verma, I. M., Retroviral vectors for gene transfer, in Microbiology-1985, American Society for Microbiology, Washington, pp. 229-232 (1985), which is incorporated by reference herein for the vectors and methods of making them. The construction of replication-defective adenoviruses has been described (Berkner et al., J. Virology 61:1213-20 (1987); Massie et. al., Mol. Cell, Biol. 6:2872-83 (1986); Haj-Ahmad et al., J. Virology 57:267-74 (1986); Davidson et al., J. Virology 61:1226-39 (1987); Zhang et. al., BioTechniques 15:868-72 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency after direct, in vivo delivery to airway epithelium, hepatocytes, vascular endothelium, CNS parenchyma and a number of other tissue sites. Other useful systems include, for example, replicating and host-restricted non-replicating vaccinia virus vectors.

Also provided are expression vectors comprising the disclosed nucleic acids, wherein the nucleic acids are operably linked to an expression control sequence. Suitable vector backbones include, for example, those routinely used in the art such as plasmids, viruses, artificial chromosomes, BACs, YACs, or PACs. Suitable expression vectors include, without limitation, plasmids and viral vectors derived from, for example, bacteriophage, baculoviruses, and retroviruses. Numerous vectors and expression systems are commercially available from such corporations as Novagen (Madison, Wis.), Ciontech (Palo Alto, Calif.), Stratagene (La Jolla, Calif.), and Invitrogen/Life Technologies (Carlsbad, Calif.). Vectors typically contain one or more regulatory regions. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns.

Particular promoters controlling transcription from vectors in mammalian host cells may be obtained from various sources, for example, the genomes of viruses such as polyoma, Simian Virus 40 (SV40), adenovirus, retroviruses, hepatitis B virus and most preferably cytomegalovirus, or from heterologous mammalian promoters, e.g., beta actin promoter or EF1 promoter, or from hybrid or chimeric promoters (e.g., cytomegalovirus promoter fused to the beta actin promoter). The early and late promoters of the SV40 virus are conveniently obtained as an SV40 restriction fragment which also contains the SV40 viral origin of replication. The immediate early promoter of the human cytomegalovirus is conveniently obtained as a HindIII E restriction fragment. Of course, promoters from the host cell or related species also are useful herein.

Enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 base pairs in length, and they function in cis. Enhancers usually function to increase transcription from nearby promoters. Enhancers can also contain response elements that mediate the regulation of transcription. While many enhancer sequences are now known from mammalian genes (globin, elastase, albumin, fetoprotein and insulin), typically one will use an enhancer from a eukaryotic cell virus for general expression. Examples of enhancers include the SV40 enhancer on the late side of the replication origin, the cytomegalovirus early promoter enhancer, the polyoma enhancer on the late side of the replication origin, and adenovirus enhancers.

Optionally, the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A particular promoter of this type is the CMV promoter. Other promoters include SV40 promoters, cytomegalovirus (plus a linked intron sequence), beta-actin, elongation factor-1 (EF-1) and retroviral vector LTR. Optionally the promoter and/or enhancer region can be inducible (e.g., chemically or physically regulated). A chemically regulated promoter and/or enhancer can, for example, be regulated by the presence of alcohol, tetracycline, a steroid, or a metal. A physically regulated promoter and/or enhancer can, for example, be regulated by environmental factors, such as temperature and light.

The vectors also can include, for example, origins of replication, scaffold attachment regions (SARs), and/or markers. A marker gene can confer a selectable phenotype, e.g., antibiotic resistance, on a cell. This marker product is used to determine if the gene has been delivered to the cell and once delivered is being expressed. Examples of marker genes include the E. coli lacZ gene, which encodes B galactosidase, green fluorescent protein (GFP), and luciferase. Examples of suitable selectable markers for mammalian cells are dihydrothlate reductase (DHFR), thymidine kinase, neomycin, neomycin analog G418, hygromycin, blasticidin, and puromycin. When such selectable markers are successfully transferred into a mammalian host cell, the transformed mammalian host cell can survive if placed under selective pressure.

In certain embodiments, LRP6 is linked to an expression tag. An expression vector can include a tag sequence designed to facilitate manipulation or detection (e.g., purification or localization) of the expressed polypeptide. Tag sequences, such as glutathione S-transferase (GST), polyhistidine (His), myc, hemagglutinin (HA), V5, IgG, T7, or FLAG™ tag (Kodak, New Haven, Conn.) sequences typically are expressed as a fusion with the encoded polypeptide. For example, LRP6 can be linked to the IgG tag. Such tags can be inserted anywhere within the polypeptide, including at either the carboxyl or amino terminus. Optionally the expression tag can be a fluorescent protein tag. Fluorescent proteins can, for example, include such proteins as green fluorescent protein ((ATP), red fluorescent protein (REP), cyan fluorescent protein (CFP), yellow fluorescent protein (YFP), and blue fluorescent protein (BFP). Fluorescent proteins can be inserted anywhere within the polypeptide, but are most preferably inserted at either the carboxyl or amino terminus.

### III. LRP6 Modulators

In certain embodiments, the LRP6 modulator is selected from the group consisting of a small molecule, a polypeptide, a nucleic acid molecule, a peptidomimetic, or a combination thereof. In a specific embodiment, the agent can be a polypeptide. The polypeptide can, for example, comprise the extracellular domain of LRP6. The polypeptide can also comprise an antibody. In another embodiment, the agent can be a nucleic acid molecule. The nucleic acid molecule can for example, be an LRP6 inhibitory nucleic acid molecule. The LRP6 or PTH1R inhibitory nucleic acid molecule can comprise a short interfering RNA (siRNA) molecule, a microRNA (miRNA) molecule, or an antisense molecule.

As used herein, an LRP6 inhibitory nucleic acid sequence can be a siRNA sequence or a miRNA sequence. A 21-25 nucleotide siRNA or miRNA sequence can, for example, be produced from an expression vector by transcription of a short-hairpin RNA (shRNA) sequence, a 60-80 nucleotide precursor sequence, which is processed by the cellular RNAi machinery to produce either an siRNA or miRNA sequence. Alternatively, a 21-25 nucleotide siRNA or miRNA sequence can, for example, be synthesized chemically. Chemical synthesis of siRNA or miRNA sequences is commercially available from such corporations as Dharmacon, Inc. (Lafayette, Colo.), Qiagen (Valencia, Calif.), and Ambion, Inc. (Austin, Tex.). An siRNA sequence preferably binds a unique sequence within the LRP6 mRNA with exact complementarity and results in the degradation of the LRP6 mRNA molecule. An siRNA sequence can bind anywhere within the mRNA molecule. An miRNA sequence preferably binds a unique sequence within the LRP6 mRNA with exact or less than exact complementarity and results in the translational repression of the LRP6 mRNA molecule. An miRNA sequence can bind anywhere within the mRNA molecule, but preferably binds within the 3′UTR of the mRNA molecule. Methods of delivering siRNA or miRNA molecules are known in the art. See, e.g., Oh and Park, Adv. Drug Deliv. Rev. 61(10):850-62 (2009); Gondi and Rao, J. Cell. Physiol. 220(2):285-91 (2009); and Whitehead et al., Nat. Rev. Drug Discos. 8(2)129-38 (2009).

As used herein, an LRP6 inhibitory nucleic acid sequence can be an antisense nucleic acid sequence. Antisense nucleic acid sequences can, for example, be transcribed from an expression vector to produce an RNA which is complementary to at least a unique portion of the LRP6 mRNA and/or the endogenous gene which encodes LRP6. Hybridization of an antisense nucleic acid molecule under specific cellular conditions results in inhibition of LRP6 protein expression by inhibiting transcription and/or translation.

The term antibody is used herein in a broad sense and includes both polyclonal and monoclonal antibodies. The term can also refer to a human antibody and/or a humanized antibody. Examples of techniques for human monoclonal antibody production include those described by Cole et al. (Monoclonal Antibodies and Cancer Therapy, Alan R. Liss, p. 77 (1985)) and by Boerner et al. (J. Immunol. 117(1):86-95 (1991)). Human antibodies (and fragments thereof) can also be produced using phage display libraries (Hoogenboom et al., J. Mol. Biol. 227:381 (1991); Marks et al., J. Mol. Biol. 222:581 (1991)). The disclosed human antibodies can also be obtained from transgenic animals. For example, transgenic mutant mice that are capable of producing a full repertoire of human antibodies, in response to immunization, have been described (see, e.g., Jakobovits et al., Proc. Natl. Acad. Sci, USA 90:2551-5 (1993); Jakobovits et al., Nature 362:255-8 (1993); Bruggermann et al., Year in Immunol. 7:33 (1993)).

In other embodiments, an LRP6 modulator is a small molecule. The term “small molecule organic compounds” refers to organic compounds generally having a molecular weight less than about 5000, 4000, 3000, 2000, 1000, 800, 600, 500, 250 or 100 Daltons, preferably less than about 500 Daltons. A small molecule organic compound may be prepared by synthetic organic techniques, such as by combinatorial chemistry techniques, or it may be a naturally-occurring small molecule organic compound.

Compound libraries may be screened for LRP6 modulators. A compound library is a mixture or collection of one or more putative modulators generated or obtained in any manner. Any type of molecule that is capable of interacting, binding or has affinity for LRP6 may be present in the compound library. For example, compound libraries screened using this invention may contain naturally-occurring molecules, such as carbohydrates, monosaccharides, oligosaccharides, polysaccharides, amino acids, peptides, oligopeptides, polypeptides, proteins, receptors, nucleic acids, nucleosides, nucleotides, oligonucleotides, polynucleotides, including DNA and DNA fragments, RNA and RNA fragments and the like, lipids, retinoids, steroids, glycopeptides, glycoproteins, proteoglycans and the like; or analogs or derivatives of naturally-occurring molecules, such as peptidomimetics and the like; and non-naturally occurring molecules, such as “small molecule” organic compounds generated, for example, using combinatorial chemistry techniques; and mixtures thereof.

A library typically contains more than one putative modulator or member, i.e., a plurality of members or putative modulators. In certain embodiments, a compound library may comprise less than about 50,000, 25,000, 20,000, 15,000, 10000, 5000, 1000, 500 or 100 putative modulators, in particular from about 5 to about 100, 5 to about 200, 5 to about 300, 5 to about 400, 5 to about 500, 10 to about 100, 10 to about 200, 10 to about 300, 10 to about 400, 10 to about 500. 10 to about 1000, 20 to about 100, 20 to about 200, 20 to about 300, 20 to about 400, 20 to about 500, 20 to about 1000, 50 to about 100, 50 to about 200, 50 to about 300, 50 to about 400, 50 to about 500, 50 to about 1000, 100 to about 200, 100 to about 300, 100 to about 400, 100 to about 500, 100 to about 1000, 200 to about 300, 200 to about 400, 200 to about 500, 200 to about 1000, 300 to about 500, 300 to about 1000, 300 to 2000, 300 to 3000, 300 to 5000, 300 to 6000, 300 to 10,000, 500 to about 1000, 500 to about 2000, 500 to about 3000, 500 to about 5000, 500 to about 6000, or 500 to about 10,000 putative modulators. In particular embodiments, a compound library may comprise less than about 50,000, 25,000, 20,000, 15,000, 10,000, 5,000, 1000, or 500 putative modulators.

A compound library may be prepared or obtained by any means including, but not limited to, combinatorial chemistry techniques, fermentation methods, plant and cellular extraction procedures and the like. A library may be obtained from synthetic or from natural sources such as for example, microbial, plant, marine, viral and animal materials. Methods for making libraries are well-known in the art. See, for example, E. R. Felder, Chimia 1994, 48, 512-541; Gallop et al., J. Med. Chem. 1994, 37, 1233-1251; R. A. Houghten, Trends Genet. 1993, 9, 235-239; Houghten et al., Nature 1991, 354, 84-86; Lam et al., Nature 1991, 354, 82-84; Carell et al., Chem, Biol. 1995, 3, 171-183; Madden et al., Perspectives in Drug Discovery and Design 2, 269-282; Cwirla et al., Biochemistry 1990, 87, 6378-6382; Brenner et al., Proc. Natl. Acad. Sci. USA 1992, 89, 5381-5383; Gordon et al., J. Med. Chem. 1994, 37, 1385-1401; Lebl et al., Biopolymers 1995, 37 177-198; and references cited therein. Compound libraries may also be obtained from commercial sources including, for example, from Maybridge, ChemNavigator.com, Timtec Corporation, ChemBridge Corporation, A-Syntese-Biotech ApS, Akos-SC, G & J Research Chemicals Ltd., Life Chemicals, Interchim S. A., and Spectrum Info. Ltd.

### IV. Functional Assays

The functional characteristics of LRP6 modulators can be tested in vitro and in vivo. LRP6 modulators may be tested for their ability to attenuate cAMP production. Assays and kits for measuring cAMP production are known in the art including, for example, cAMP-GLO® Assay (Promega Corp. (Madison, Wis.)); CatchPoint cAMP Detection Assay Kit (Molecular Devices, LLC (Sunnyvale, Calif.)); LANCE cAMP assay (PerkinElmer, Inc. (Waltham, Mass.); HTRF technology-based cAMP assay (Cisbio US (Bedford, Mass.)); and EIA kit (Amersham Pharmacia Biotech (Piscataway, N.J.)). In other embodiments, reporter-gene systems for the study of ligand activity at Gα₂ can be used. See, e.g., Kent et al., 10 J. BIOMOL. SCREEN 437-46 (2005); and Hill et al., 5 CURB. OPIN. PHARMACOL. 526-32 (2001).

Modulators can also be tested for the ability to interfere with LRP6's ability to bind its natural ligands including, but not limited to DKK1 and Wnt pathway members DKK1 (dickkopf 1), DKK2, DKK4, SOST1, SOSD1 (USAG1), sFRP (soluble Fzd-related protein) 1-4, Wise, or Wnt ligands), or to modulate such biological processes as β-catenin phosphorylation and degradation, tumor cell growth, apoptosis, regulation of bone mineral density, and insulin secretion.

LRP6 binding to ligands can be detected using Biacore® by immobilizing ligands to a solid support and detecting soluble LRP6 binding thereto. Alternatively, LRP6 can be immobilized, and the ligand binding thereto can be detected. LRP6/ligand binding can also be analyzed by ELISA (e.g., by detecting LRP6 binding to immobilized ligands), or by fluorescence resonance energy transfer (FRET). To perform FRET, fluorophore-labeled LRP6 binding to ligands in solution can be detected (see, for example, U.S. Pat. No. 5,631,169).

LRP6-ligand binding can also be detected via “liquid binding” methods, i.e., measuring affinity in liquid setting, instead of in an immobilized environment. Such methods are offered by Roche. LRP6-ligand binding can also be detected by coimmunoprecipitation (Lagace et al., 2006 J. Clin. Inv. 116(10:2995-3005). To examine LRP6-ligand binding in this manner, HepG2 cells are cultured in sterol-depleted medium for 18 hours. Purified LRP6 is added to the medium in the presence of 0.1 mM chloroquine and the cells are incubated for one hour. Cells are lysed in mild detergent (1% digitonin w/vol). LRP6 or a ligand is immunoprecipitated from cell lysates, separated by SDS-PAGE, and immunoblotted to detect the presence of coimmunoprecipitated the ligand or LRP6, respectively (Lagace et al., 2006 J. Clin. Inv. 116(11):2995-3005). These assays may be conducted with a mutant form of LRP6 that binds to the ligand with a higher avidity (Lagace et al., 2006, supra).

LRP6 modulators can be tested for the ability to increase or decrease ligand levels within the cells. For example, cells are cultured in sterol-depleted medium (DMEM supplemented with 100 U/ml penicillin, 100 μg/ml streptomycin sulfate, and 1 g/l glucose, 5% (vol/vol) newborn calf lipoprotein-deficient serum (NCLPDS), 10 μM sodium compactin, and 50 μM sodium mevalonate) for 18 hours to induce ligand expression. Purified LRP6 (about 5 μg/ml) is added to the medium. Ligand levels in cells harvested at 0, 0.5, 1, 2, and 4 hours after addition of LRP6 is determined (Lagace et al., 2006 J. Clin. Inv. 116(11):2995-3005). Ligand levels can be determined by flow cytometry. FRET, immunoblotting, or other means.

LRP6 modulators can be tested for the ability to increase or decrease cAMP production, LRP6-ligand binding, LRP6 phosphorylation, β-catenin stabilization, reporter gene or target gene expression. LRP6-ligand binding can be detected by production of cell membrane preparations containing LRP6 and tested for ability to bind the preparations compared to cell membrane preparations not containing LRP6, or by FACS analysis of cells with endogenous LRP6 or cells with overexpressed LRP6.

LRP6 modulators (e.g., anti-LRP6 antibodies) may be tested by their ability to inhibit growth of tumors in mice. Inhibition of tumor growth within tumor xenograft models (e.g., SCID xenograft models, orthotopic xenograft models, etc.) is an environment in which LRP6 modulators may be tested by their ability to inhibit tumor growth. LRP6 modulators may also be tested by their ability to inhibit growth of tumors in vitro, such as inhibition of colony formation in soft agar.

### V. Methods of Using LRP6 Modulators

The LRP6 modulators described herein have in vitro and in vivo diagnostic and therapeutic utilities. For example, these molecules can be administered to cells in culture, e.g., in vitro or in vivo, or in a subject, e.g., in vivo, to treat, prevent or diagnose a variety of LRP6-mediated diseases, disorders or conditions. LRP6 modulators are particularly suitable for treating human patients suffering from “Wnt signaling-related disorders,” meaning those diseases and conditions associated with aberrant Wnt signaling. Aberrant upregulation of Wnt signaling is associated with cancer, osteoarthritis, and polycystic kidney disease, which conditions would be particularly amendable to treatment by the administration of antagonizing LRP modulators. Conversely, aberrant downregulation of Wnt signaling has been linked to osteoporosis, obesity, diabetes, and neuronal degenerative diseases, which conditions would be particularly amendable to treatment by the administration of agonizing LRP modulators.

“Wnt signaling-related cancers” which are amenable to treatment by the administration of the LRP6 modulators of the invention include but are not limited to colon cancers (e.g., colorectal carcinomas (CRCs)), melanoma, breast cancer, liver cancer, lung cancer, gastric cancer, malignant medulloblastoma and other primary CNS malignant neuroectodermal tumors, rhabdornyosarcoma, gut-derived tumors (including but not limited to cancer of the esophagus, stomach, pancreas, and biliary duct system), and prostate and bladder cancers.

In a related fashion, the LRP6 antagonizing modulators of the present invention are capable of inhibiting the growth of a tumor cell, or of inducing apoptosis in, or inhibiting angiogenesis of, a tumor cell. By way of example, a tumor cell can be contacted with an antagonizing LRP6 modulator (e.g., an LRP6 modulator including an antigen binding portion of an antibody that specifically binds to an LRP6), thereby preventing Wnt pathway signal transduction within the tumor cell via stabilizing the β-catenin destruction complex and engendering β-catenin phosphorylation and degradation.

The LRP6 modulators described herein are particularly suitable for treating human patients suffering from bone-related disorders, such as in the case of fractures, disorders in which bone mineral density (BMD) is abnormally and/or pathologically high relative to healthy subjects, and disorders in which bone mineral density (BMD) is abnormally and/or pathologically low relative to healthy subjects.

Disorders characterized by high BMD, which would be amenable to treatment via the administration of LRP6 modulators, include but are not limited to sclerosteosis, Van Buchem disease, bone overgrowth disorders, and Simpson-Golabi-Behmel syndrome (SGBS). By way of example, said antagonizing LRP6 modulators (e.g., an LRP6 modulator including an antigen binding portion of an antibody that specifically binds to an LRP6) would be administered to a subject in an amount effective to lower bone mineral density.

Disorders characterized by low BMD and/or bone fragility, which would be amenable to treatment via the administration of LRP6 modulators include, but are not limited to, primary and secondary osteoporosis, osteopenia, osteomalacia, osteroporosis-pseudoglioma syndrome (OPPG), osteogenesis imperfecta (OI), avascular necrosis (osteonecrosis), fractures and implant healing (dental implants and hip implants), and bone loss due to other disorders (e.g., associated with HIV infection, cancers, or arthritis). In particular embodiments, the LRP6 modulators can bind to LRP6 and engender the dissolution of the β-catenin destruction complex, thereby permitting β-catenin stabilization, translocation to the nucleus, and engagement of transcription factors, and thereby raising bone mineral density in the process.

In one embodiment, the modulators of the invention cat be used to detect levels of LRP6. This can be achieved, for example, by contacting a sample (such as an in vitro sample) and a control sample with the LRP6 modulator under conditions that allow for the formation of a complex between the modulator and LRP6. Any complexes formed between the molecule and LRP6 are detected and compared in the sample and the control. For example, standard detection methods, well known in the art, such as ELISA and flow cytometric assays, can be performed using the compositions of the invention.

Accordingly, in one aspect, the invention further provides methods for detecting the presence of LRP6 (e.g., hLRP6) in a sample, or measuring the amount of LRP6, comprising contacting the sample, and a control sample, with an LRP6 modulator (e.g., an antibody) of the invention, under conditions that allow for formation of a complex between the antibody or portion thereof and LRP6. The formation of a complex is then detected, wherein a difference in complex formation between the sample compared to the control sample is indicative of the presence of LRP6 in the sample.

Also within the scope of the invention are kits comprising the compositions of the invention and instructions for use. In one embodiment, the kit comprises an anti-LRP6 antibody. The kit can further contain a least one additional reagent, or one or more additional antibodies (e.g., an antibody having a complementary activity which binds to an epitope on the target antigen distinct from the first antibody). Kits typically include a label indicating the intended use of the contents of the kit. The term label includes any writing, or recorded material supplied on or with the kit, or which otherwise accompanies the kit.

### VI. Pharmaceutical Compositions and Administration

Accordingly, a pharmaceutical composition of the present invention may comprise an effective amount of an LRP6 modulator. As used herein, the term “effective,” means adequate to accomplish a desired, expected, or intended result. More particularly, an “effective amount” or a “therapeutically effective amount” is used interchangeably and refers to an amount of an LRP6 modulator, perhaps in further combination with yet another therapeutic agent, necessary to provide the desired “treatment” (defined herein) or therapeutic effect, e.g., an amount that is effective to prevent, alleviate, treat or ameliorate symptoms of a disease or prolong the survival of the subject being treated. In particular embodiments, the pharmaceutical compositions of the present invention are administered in a therapeutically effective amount to treat patients suffering from an LRP6-mediated disease, disorder or condition. As would be appreciated by one of ordinary skill in the art, the exact low dose amount required will vary from subject to subject, depending on age, general condition of the subject, the severity of the condition being treated, the particular compound and/or composition administered, and the like. An appropriate “therapeutically effective amount” in any individual case can be determined by one of ordinary skill in the art by reference to the pertinent texts and literature and/or by using routine experimentation.

The pharmaceutical compositions of the present invention are in biologically compatible form suitable for administration in vivo for subjects. The pharmaceutical compositions can further comprise a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which an LRP6 modulator is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water may be a carrier when the pharmaceutical composition is administered orally. Saline and aqueous dextrose may be carriers when the pharmaceutical composition is administered intravenously. Saline solutions and aqueous dextrose and glycerol solutions may be employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried slim milk, glycerol, propylene, glycol, water, ethanol and the like. The pharmaceutical composition may also contain minor amounts of wetting or emulsifying agents, or pH buffering agents.

The pharmaceutical compositions of the present invention can take the form of solutions, suspensions, emulsions, tablets, pills, capsules, powders, sustained-release formulations and the like. The composition can be formulated as a suppository, with traditional binders and carriers such as triglycerides. Oral formulation may include standard carriers such as pharmaceutical grades of mannitol, lactose, starch, magnesium stearate, sodium saccharine, cellulose, magnesium carbonate, etc. In a specific embodiment, a pharmaceutical composition comprises an effective amount of an LRP6 modulator together with a suitable amount of a pharmaceutically acceptable carrier so as to provide the form for proper administration to the patient. The formulation should suit the mode of administration.

The pharmaceutical compositions of the present invention may be administered by any particular route of administration including, but not limited to oral, parenteral, subcutaneous, intramuscular, intravenous, intrarticular, intrabronchial, intraabdominal, intracapsular, intracartilaginous, intracavitary, intracelial intracelebellar, intracerebroventricular, intracolic, intracervical, intragastric, intrahepatic, intramyocardial, intraosteal, intraosseous, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, iontophoretic means, or transdermal means. Most suitable routes are oral administration or injection. In certain embodiments, subcutaneous injection is preferred.

In general, the pharmaceutical compositions comprising an LRP6 modulator may be used alone or in concert with other therapeutic agents at appropriate dosages defined by routine testing in order to obtain optimal efficacy while minimizing any potential toxicity. The dosage regimen utilizing a pharmaceutical composition of the present invention may be selected in accordance with a variety of factors including type, species, age, weight, sex, medical condition of the patient; the severity of the condition to be treated; the route of administration; the renal and hepatic function of the patient; and the particular pharmaceutical composition employed. A physician of ordinary skill can readily determine and prescribe the effective amount of the pharmaceutical composition (and potentially other agents including therapeutic agents) required to prevent, counter, or arrest the progress of the condition.

Optimal precision in achieving concentrations of the therapeutic regimen (e.g., pharmaceutical compositions comprising an LRP6 modulator, optionally in combination with another therapeutic agent) within the range that yields maximum efficacy with minimal toxicity may require a regimen based on the kinetics of the pharmaceutical composition's availability to one or more target sites. Distribution, equilibrium, and elimination of a pharmaceutical composition may be considered when determining the optimal concentration for a treatment regimen. The dosages of a pharmaceutical composition disclosed herein may be adjusted when combined to achieve desired effects. On the other hand, dosages of the pharmaceutical compositions and various therapeutic agents may be independently optimized and combined to achieve a synergistic result wherein the pathology is reduced more than it would be if either was used alone.

In particular, toxicity and therapeutic efficacy of a pharmaceutical composition disclosed herein may be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effect is the therapeutic index and it may be expressed as the ratio LD₅₀/ED₅₀. Pharmaceutical compositions exhibiting large therapeutic indices are preferred except when cytotoxicity of the composition is the activity or therapeutic outcome that is desired. Although pharmaceutical compositions that exhibit toxic side effects may be used, a delivery system can target such compositions to the site of affected tissue in order to minimize potential damage to uninfected cells and, thereby, reduce side effects. Generally, the pharmaceutical compositions of the present invention may be administered in a manner that maximizes efficacy and minimizes toxicity.

Data obtained from cell culture assays and animal studies may be used in formulating a range of dosages for use in humans. The dosages of such compositions lie preferably within a range of circulating concentrations that include the ED₅₀ with little or no toxicity. The dosage may vary within this range depending upon the dosage form employed and the route of administration utilized. For any composition used in the methods of the invention, the therapeutically effective dose may be estimated initially from cell culture assays. A dose may be formulated in animal models to achieve a circulating plasma concentration range that includes the IC₅₀ (the concentration of the test composition that achieves a half-maximal inhibition of symptoms) as determined in cell culture. Such information may be used to accurately determine useful doses in humans. Levels in plasma may be measured, for example, by high performance liquid chromatography.

Moreover, the dosage administration of the compositions of the present invention may be optimized using a pharmacokinetic/pharmacodynamic modeling system. For example, one or more dosage regimens may be chosen and a pharmacokinetic/pharmacodynamic model may be used to determine the pharmacokinetic/pharmacodynamic profile of one or more dosage regimens. Next, one of the dosage regimens for administration may be selected which achieves the desired pharmacokinetic/pharmacodynamic response based on the particular pharmacokinetic/pharmacodynamic profile. See WO 00/67776, which is entirely expressly incorporated herein by reference.

More specifically, the pharmaceutical compositions may be administered in a single daily dose, or the total daily dosage may be administered in divided doses of two, three, or four times daily. In the case of oral administration, the daily dosage of the compositions may be varied over a wide range from about 0.1 ng to about 1,000 mg per patient, per day. The range may more particularly be from about 0.001 ng/kg to 10 mg/kg of body weight per day, about 0.1-100 μg, about 1.0-50 μm or about 1.0-20 mg per day for adults (at about 60 kg).

The daily dosage of the pharmaceutical compositions may be varied over wide range from about 0.1 ng to about 1000 mg per adult human per day. For oral administration, the compositions may be provided in the form of tablets containing from about 0.1 ng to about 1000 mg of the composition or 0.1, 0.2, 0.5, 1.0, 2.0, 5.0, 10.0, 15.0, 100, 150, 200, 250, 300, 350, 400, 450, 500, 550, 600, 650, 700, 800, 900, or 1000 milligrams of the composition for the symptomatic adjustment of the dosage to the patient to be treated. An effective amount of the pharmaceutical composition is ordinarily supplied at a dosage level of from about 0.1 ng/kg to about 20 mg/kg of body weight per day. In one embodiment, the range is from about 0.2 ng/kg to about 10 mg/kg of body weight per day. In another embodiment, the range is from about 0.5 ng/kg to about 10 mg/kg, of body weight per day. The pharmaceutical compositions may be administered on a regimen of about 1 to about 10 times per day.

In the case of injections, it is usually convenient to give by an intravenous route in an amount of about 0.0001 μg-30 mg, about 0.01 μg-20 mg or about 0.01-10 mg per day to adults (at about 60 kg). In the case of other animals, the dose calculated for 60 kg may be administered as well.

Doses of a pharmaceutical composition of the present invention can optionally include 0.0001 μg to 1,000 mg/kg/administration, or 0.001 μg to 100.0 mg/kg/administration, from 0.01 μg to 10 mg/kg/administration, from 0.1 μg to 10 mg/kg/administration, including, but not limited to, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100-500 mg/kg/administration or any range, value or fraction thereof, or to achieve a serum concentration of 0,1, 0.5, 0.9, 1,0, 1,1, 1.2, 1.5, 1.9, 2.0, 2.5, 2,9, 3.0, 3.5, 3.9, 4,0, 4.5, 4.9, 5,0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 119, 14.0, 14.5, 4.9, 5.0, 5.5, 5.9, 6.0, 6.5, 6.9, 7.0, 7.5, 7.9, 8.0, 8.5, 8.9, 9.0, 9.5, 9,9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19, 19.5, 19.9, 20, 20.5, 20.9, 21, 22, 23, 24, 25, 26, 27, 28, 29, 30. 35, 40, 45, 50, 55, 60, 65, 70, 75, 80, 85, 90, 96, 100, 200, 300, 400, 500, 600, 700, 800, 900, 1000, 1500, 2000, 2500, 3000, 3500, 4000, 4500, and/or 5000 μg/ml serum concentration per single or multiple administration or any range, value or fraction thereof.

As a non-limiting example, treatment of subjects can be provided as a one-time or periodic dosage of a composition of the present invention 0.1 ng to 100 mg/kg such as 0.0001, 0.001, 0.01, 0.1 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively or additionally, at least one of week 1, 2, 3, 4, 5, 6, 7, 8, 9, 10, 11, 12, 13, 14, 15, 16, 17, 18, 19, 20, 21, 22, L 24, 25, 26, 27, 28, 30, 31, 32, 33, 34 35, 36, 37, 38, 39, 40, 41, 42, 43, 44, 45, 46, 47, 48, 49, 50, 51, or 52, or alternatively or additionally, at least one of 1, 2, 3, 4, 5, 6, 7, 8, 9, 10, 11, 12, 13, 14, 15, 16, 17, 18, 19, or 20 years, or any combination thereof, using single, infusion or repeated doses.

Specifically, the pharmaceutical compositions of the present invention may be administered at least once a week over the course of several weeks. In one embodiment, the pharmaceutical compositions are administered at least once a week over several weeks to several months. In another embodiment, the pharmaceutical compositions are administered once a week over four to eight weeks. In yet another embodiment, the pharmaceutical compositions are administered once a week over four weeks.

More specifically, the pharmaceutical compositions may be administered at least once a day for about 2 days, at least once a day for about 3 days, at least once a day for about 4 days, at least once a day for about 5 days, at least once a day for about 6 days, at least once a day or about 7 days, at least once a day for about 8 days, at least once a day for about 9 days, at least once a day for about 10 days, at least once a day for about 11 days, at least once a day for about 12 days, at least once a day for about 13 days, at least once a day for about 14 days, at least once a day for about 15 days, at least once a day for about 16 days, at least once a day for about 17 days, at least once a day for about 18 days, at least once a day for about 19 days, at least once a day for about 20 days, at least once a day for about 21 days, at least once a day for about 22 days, at least once a day for about 23 days, at least once a day for about 24 days, at least once a day for about 25 days, at least once a day for about 26 days, at least once a day for about 27 days, at least once a day for about 28 days, at least once a day for about 29 days, at least once a day for about 30 days, or at least once a day for about 31 days.

Alternatively, the pharmaceutical compositions may be administered about once every day, about once every 2 days, about once every 3 days, about once every 4 days, about once every 5 days, about once every 6 days, about once every 7 days, about once every 8 days, about once every 9 days, about once every 10 days, about once every 11 days, about once every 12 days, about once every 13 days, about once every 14 days, about once every 15 days, about once every 16 days, about once every 17 days, about once every 18 days, about once every 19 days, about once every 20 days, about once every 21 days, about once every 22 days, about once every 23 days, about once every 24 days, about once every 25 days, about once every 26 days, about once every 27 days, about once every 28 days, about once every 29 days, about once every 30 days, or about once every 31 days.

The pharmaceutical compositions of the present invention may alternatively be administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, about once every 12 weeks, about once every 13 weeks, about once every 14 weeks, about once every 15 weeks, about once every 16 weeks, about once every 17 weeks, about once every 18 weeks, about once every 19 weeks, about once every 20 weeks.

Alternatively, the pharmaceutical compositions of the present invention may be administered about once every month, about once every 2 months, about once every 3 months, about once every 4 months, about once every 5 months, about once every 6 months, about once every 7 months, about once every 8 months, about once every 9 months, about once every 10 months, about once every 11 months, or about once every 12 months.

Alternatively, the pharmaceutical compositions may be administered at least once a week for about 2 weeks, at least once a week for about 3 weeks, at least once a week for about 4 weeks, at least once a week for about 5 weeks, at least once a week for about 6 weeks, at least once a week for about 7 weeks, at least once a week for about 8 weeks, at least once a week for about 9 weeks, at least once a week for about 10 weeks, at least once a week for about 11 weeks, at least once a week for about 12 weeks, at least once a week for about 13 weeks, at least once a week for about 14 weeks, at least once a week 1 hr about 15 weeks, at least once a week for about 16 weeks, at least once a week for about 17 weeks, at least once a week for about 18 weeks, at least once a week for about 19 weeks, or at least once a week for about 20 weeks.

Alternatively the pharmaceutical compositions may be administered at least once a week for about 1 month, at least once a week for about 2 months, at least once a week for about 3 months, at least once a week for about 4 months, at least once a week for about 5 months, at least once a week for about 6 months, at least once a week for about 7 months, at least once a week for about 8 months, at least once a week for about 9 months, at least once a week for about 10 months, at least once a week for about 11 months, or at least once a week for about 12 months.

The pharmaceutical compositions may further be combined with one or more additional therapeutic agents. The second therapeutic agent can be a chemotherapeutic, in the case of cancers; an insulin secretion enhancer, such as nateglinides or repaglinides, in the case of diabetes or other metabolic disorders; and agents capable of increasing bone density, such as calcium, vitamin D, or bisphosphonates, in the case of bone-related disorders. A combination therapy regimen may be additive, or it may produce synergistic results (e.g., increases in bone mineral density, or in insulin secretion, or in apoptosis of cancer cells greater than expected for the combined use of the two agents).

The compositions can be administered simultaneously or sequentially by the same or different routes of administration. The determination of the identity and amount of the pharmaceutical compositions for use in the methods of the present invention can be readily made by ordinarily skilled medical practitioners using standard techniques known in the art. In specific embodiments, an LRP6 modulator of the present invention can be administered in combination with an effective amount of an another therapeutic agent, depending on the disease or condition being treated.

In various embodiments, the LRP6 modulator of the present invention in combination with an another therapeutic agent may be administered at about the same time, less than 1 minute apart, less than 2 minutes apart, less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In particular embodiments, two or more therapies are administered within the same patent visit.

In certain embodiments, the LRP6 modulator of the present invention in combination with another therapeutic agent are cyclically administered. Cycling therapy involves the administration of a first therapy (e.g., the LRP6 modulator) for a period of time, followed by the administration of a second therapy (e.g., another therapeutic agent) for a period of time, optionally, followed by the administration of perhaps a third therapy for a period of time and so forth, and repeating this sequential administration, e.g., the cycle, in order to reduce the development of resistance to one of the therapies, to avoid or reduce the side effects of one of the therapies, and/or to improve the efficacy of the therapies. In certain embodiments, the administration of the combination therapy of the present invention may be repeated and the administrations may be separated by at least 1 day, 2 days, 3 days, 5 days, 10 days, 15 days, 30 days, 45 days, 2 months, 75 days, 3 months, or at least 6 months.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Materials and Methods

cDNA Constructs. The complementary DNA (cDNA) sequence encoding PTH1R was subcloned into the plasmid pCDNA1 (52), the eDNA sequence encoding Gαₛ was subcloned into pCMV5B, the Gαₛ-GFP cDNA was subcloned into the plasmid pCDNA3 (53), whereas the eDNA encoding VSVG-tagged LRP6 was subcloned into the plasmid pCS2+ (26). siRNAs specific for GFP (sc-45924), mouse LRP5 (sc-149050), human LRP5 (sc-43900), mouse LRP6 (sc-37234), and human LRP6 (sc-37233) were purchased from Santa Cruz Biotechnology Inc. cDNAs encoding VSVG-tagged LRP6^{mGSK3} and LRP6^{mPKA} were generated by mutagenesis of the codons encoding Ser¹⁵⁴⁴ or Thr¹⁵⁴⁸ to a codon encoding alanine. Sequences encoding the cytoplasmic domain of LRP5 (amino acid residues 192 to 566, LRP5C) and the cytoplasmic domain of LRP6 (amino acid residues 461 to 593, LRP6C) were fused with cDNA encoding GST in the pGEX-KG prokaryotic gene fusion vector (Pharmacia). The mutants GST-LRP6^{mGSK3} and GST-LRP6C^{mPKA} were generated by primer-mediated polymerase chain reaction (PCR) mutagenesis and verified by DNA sequencing. Sequences encoding FLAG-tagged Gαₛ and HA-tagged Gβ₁ at their N termini were subcloned into the plasmids pCMV5B and pCDNA3, respectively. The sequence of the siRNA specific for human axin (5′-UGCCAAGAAGGCUGAGUCG-3′) (SEQ ID NO:3) was designed as described previously (54).

Detection of cAMP The amount of cAMP produced by cells in response to PTH or other GPCR agonists was determined with a ³H-labeled cAMP assay, as described previously (55, 56). Briefly, cells were plated into six wells and incubated with 25 mM Hepes and [3H]adenine (2 mCi/ml) at 37° C. for 2 hours. After the isotope was removed by washing, cells were stimulated for 20 to 30 min at 37° C. with PTH or other GPCR agonists in the presence of 1 mM IBMX (3-isobutyl-1-methylxanthine) and 0.4 mM ascorbate. Forskolin (10 mM) was used as a positive control. To terminate stimulation, the media was aspirated, 1 ml of 5% trichloroacetic acid added to each well, and the plates stored at 4° C. overnight to collect the extracts. Separation of [³H]cAMP from [³H]ATP (adenosine 5′-triphosphate) was performed by chromatography on Dowex and alumina columns, according to the method of Salomon et al. (57). The amount of cAMP formed from ATP was calculated as follows: % conversion=[³H]cAMP/([³H]cAMP+[³H]ATP)×100 per 20 or 30 min.

Detection of the Temporal Dynamics of cAMP Signaling in Living Cells. The binding of cAMP to Epac induces a conformational change that liberates the catalytic domain of Epac from intrasubunit allosteric inhibition (42). A chimeric protein (ICUE3) generated by fusing the N terminus of a truncated form of Epac to enhanced CFP (ECFP) and the C terminus of Epac to citrine, an improved version of yellow fluorescent protein (YFP), was produced as previously described (43). Sandwiching these conformationally responsive elements of Epac between a FRET pair (citrine and ECFP) enables cAMP production and degradation to be monitored by detecting changes in FRET. Therefore, changes in cAMP could be monitored in real time in single live cells. Experiments were performed as previously described (41). HEK 293 cells plated on glass-bottom petri dishes (MatTek Corporation) were washed twice with buffer containing Hanks' balanced salt solution (HBSS) and placed on a Zeiss Axiovert 200M inverted microscope with a cooled charge-coupled device camera (MicroMAX BFT512, Roper Scientific). Cells were maintained in HBSS-containing buffer in the dark with the addition of PTH, isoproterenol, or forskolin as indicated in the figure legends. Cells were imaged with METAFLUOR 6.2 software (Universal Imaging) as the ratio between emission at 535 nm with a 535DF25 band-pass filter for citrine and emission at 475 nm with a 475DF40 band-pass filter for ECFP, upon excitation at 420 nm with a 420DF20 band-pass filter. Images were acquired every 30 s with an exposure time of 100 to 500 ms, which resulted in negligible photobleaching over a 30-min observation time. Fluorescent images were corrected for background by subtracting the autofluorescence intensities of untransfected cells (or the background with no cells) from the emission intensities of fluorescent cells that contained reporter constructs. The ratios of cyan-to-yellow emissions were then calculated at different time points and normalized by dividing all of the ratios by the emission ratio just before stimulation, thereby setting the basal emission ratio to 1. Citrine was photobleached at the end of the experiment by intense illumination with a 525DF40 filter. The fluorescence intensities of ECFP before (F_{da}) and after (F_{d}) citrine photobleaching and the equation E=1−(F_{da}/F_{d}) were used to calculate the FRET efficiency.

Cell Culture, Transfections, Coimmunoprecipitation, and Western Blotting Analysis. HEK293,UMR-106,C2C12, and MEF cells were maintained in Dulbecco's modified Eagle's medium (DMEM) with 10% fetal calf serum (FCS). Transfections were performed with Lipofectamine reagent (Invitrogen). Immunoprecipitations and Western blotting analysis of cell lysates were performed as described previously (37). Briefly, cells were lysed in immunoprecipitation buffer [50 mM tris-HCl (pH7.5), 150 mM NaCl, 1% Triton X-100, and 0.5% sodium deoxycholate containing protease inhibitors. The lysates were subjected to immunoprecipitation by incubation with the appropriate antibodies followed by absorption on protein G-Sepharose. The immunoprecipitates were separated by SDS-polyacrylamide gel electrophoresis (SDS-PAGE) and transferred onto a nitrocellulose membrane. Western blots were characterized with the SuperSignal West Femto Substrate system (Pierce, Thermo Fisher Scientific Inc.). Primary antibodies used for Western blotting analysis and immunoprecipitations included mouse antibody against the FLAG tag (M2, Sigma-Aldrich), rabbit antibody against VSVG (Covance), mouse antibody against the HA tag (16B12, Covance), rabbit antibody against PTH1R, (PRB-640P, Covance), rabbit antibodies against CREB and phosphorylated CREB (pCREB) (both from Upstate), and rabbit antibody against Gαₛ and mouse antibody against Gb1 (both from Santa Cruz Biotechnology).

GST Fusion Protein Expression, Pull-Down Assays, and In Vitro Kinase Assays. The GST-tagged cytoplasmic domains of LRP5 (amino acid residues 192 to 566) and LRP6 (residues 461 to 593) and its mutant fragments were expressed in BL21 (DE3) Escherichia coli cells. The GST fusion proteins were then purified, and pull-down assays were performed as outlined previously (58). In vitro kinase assays were performed by the addition of bead-bound GST fusion proteins to buffer that contained 50 mM tris-HCl (pH 7.5), 10 mM MgCl2, and 200 mCi [γ-³²P]ATP, together with the catalytic subunits of PKA, GSK-3β, or both in a volume of 30 ml. Reaction mixtures were incubated at room temperature for 30 min before the samples were resolved by 10% SDS-PAGE. The amount of protein-associated radiolabel was determined by PhosphorImager analysis (Molecular Dynamics).

Immunofluorescence Detection of the Localization of Gαs, Gb1, and PTH1R. Cells stably expressing GFP-Gαs (53), GFP-Gb1 (59), or CFP-PTH1R were fixed with 100% methanol, permeabilized with 0.5% Triton X-100, and blocked in 2% bovine serum albumin in tris-buffered saline (TBS) containing 0.1% Tween 20. Digital pictures were taken with an Olympus IX TRTNOC camera under an Olympus IX70 Inverted Research Microscope with a 10× objective lens and Hoffman modulation contrast at room temperature and processed with MagnaFire SP imaging software (Optronics). The numbers of cells with membrane-bound fluorescent protein or cytosolic fluorescent protein were quantified. For each treatment, 100 cells in three different slides were analyzed. The ratios of the number of cells with membrane bound fluorescent protein or cytosolic fluorescent protein to the total cell numbers were calculated and expressed as a mean percentage±SD.

Isolation and Culture of Primary Mouse Osteoblasts. Primary osteoblasts were obtained through serial digestion of calvaria of newborn mice in collagenase type I (1.8 mg/ml; Worthington Biochemical Corp.), as described previously (60). Briefly, calvaria from mice were digested in 10 ml of digestion solution for 15 min at 37° C. with constant agitation. The supernatant was collected and replaced with fresh collagenase, and the digestion was repeated an additional four times. Final digestion solutions containing the osteoblasts were collected and centrifuged at 500 g for 10 min at room temperature, and the osteoblasts were cultured in a-minimal essential medium (a-MEM) containing 10% fetal bovine serum (FBS) and 1% penicillin and streptomycin at 37° C. in a humidified incubator supplied with 5% CO₂.

Generation of Conditional lrp6 Knockout Mice and Bone Immunohistochemistry Analysis. The genotype of transgenic mice was determined by PCR analysis of genomic DNA isolated from mouse tails. Mice with osteoblast-specific inactivation of lrp6 (lrp6 knockouts) were generated by crossing OC-Cre mice with mice homozygous for a foxed lrp6 allele. All mice were given a standard chow diet and water. Procedures involving mice were approved by the Institutional Animal Care and Use Program of the Johns Hopkins University. The mice were killed, and femora were resected and fixed in 10% buffered formalin for 48 hours, decalcified in 10% EDTA (pH 7.0) for 20 days, and embedded in paraffin. Immunostaining was performed with a standard protocol, as described previously (61). Sections were incubated with a 1:50 dilution of antibody against Gαₛ (Santa Cruz Biotechnology Inc.) overnight at 4° C. A horseradish peroxidase (HRP)-streptavidin detection system (Dako) was used to detect immunoreactivity, followed by counterstaining with methyl green (Sigma-Aldrich).

### Results

### Example 1

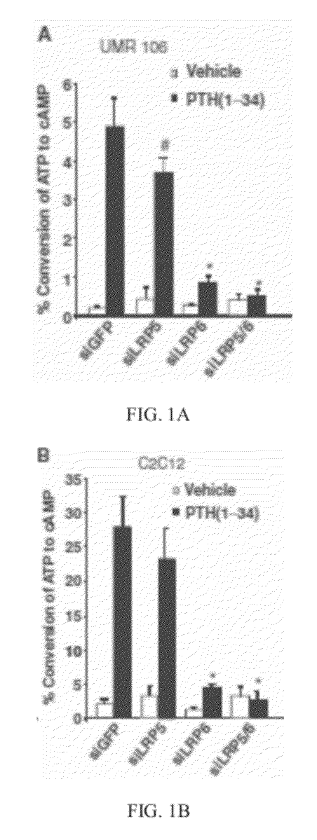

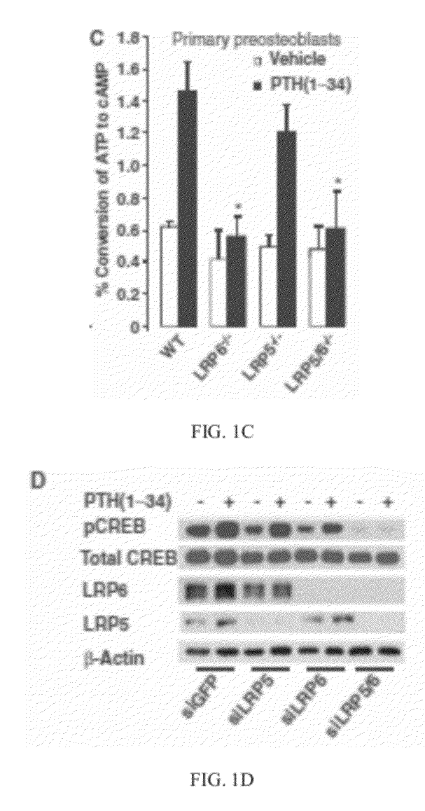

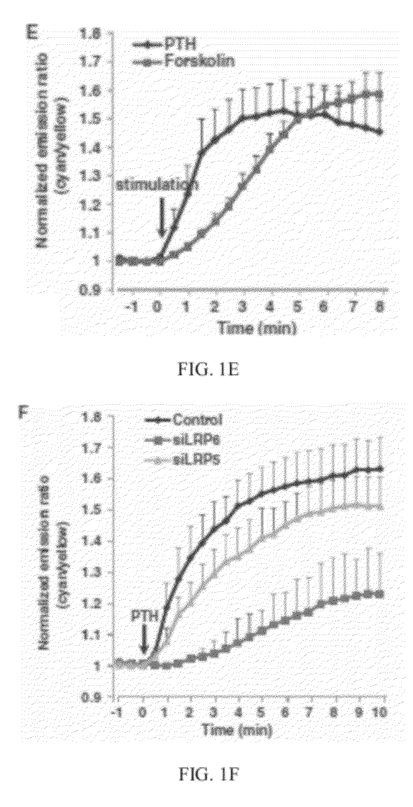

LRP6 is Required for PTH-Dependent Generation of cAMP. To examine the role of LRP6 in PTH dependent cAMP production, small interfering RNA (siRNA) were used to knock down LRP6 or LRP5 in UMR-106 rat osteosarcoma cells (FIG. 1A) and C2C12 pluripotent mouse osteoprogenitor cells (FIG. 1B). The cells were then treated with PTH(1-34), a C-terminal-truncated synthetic analog of PTH that has an anabolic effect on bone formation in humans (39). Knockdown of LRP6 inhibited the PTH (1-34)-stimulated accumulation of cAMP in both cell types, whereas knockdown of LRP5 did not affect cAMP production in C2C12 cells (FIG. 1B) and only moderately inhibited cAMP accumulation in response to PTH in UMR-106 cells (FIG. 1A). To further confirm the requirement for LRP6 in PTH-induced cAMP accumulation in primary osteoblasts, calvarial preosteoblasts were isolated from lrp6flox/flox or lrp6 flox/flox; lrp5−/− mice (40). The lrp6 gene was deleted by adenovirus-mediated expression of the Cre recombinase gene. PTH-stimulated cAMP accumulation in primary preosteoblasts was inhibited by deletion of LRP6, but not of LRP5 (FIG. 1C).

Because PKA can be activated by cAMP that accumulates in response to the stimulation of Gₛ-coupled GPCRs, the extent of PTH-stimulated phosphorylation of cAMP-responsive element (CRE)-binding protein (CREB), a transcription factor that is a direct downstream target of PKA (37, 38), was examined. Simultaneous knockdown of LRP5 and LRP6 inhibited the PTH-induced phosphorylation of CREB in UMR-106 cells (FIG. 1D), Phosphorylation of CREB was partially inhibited when LRP6 alone was knocked down, which suggested that LRP5 might also play a role in PTH activated cAMP-PKA signaling specifically in UMR-106 cells. To assess whether knockdown of LRP6 affected the temporal dynamics of cAMP production, the kinetics of cAMP generation in live cells were monitored with Epac (ICUE3), a fluorescence resonance energy transfer (FRET)-based biosensor of cAMP production (41-43). PTH(1-34) induced a rapid change in the ratio of cyan-to-yellow emissions (an indication of cAMP production) within 30 s, which reached a plateau within ˜4 to 6 min, whereas a slower response to forskolin (FIG. 1E), a direct activator of AC (44), was observed. Cells in which LRP6 was knocked down exhibited a minimum response to PTH, whereas cells in which LRP5 was knocked down had a slightly smaller response to PTH than that of the control cells (FIG. 1F). These results indicate that LRP6 is required for PTH-activated cAMP-PKA signaling and that LRP5 plays a relatively minor role in this pathway.

### Example 2

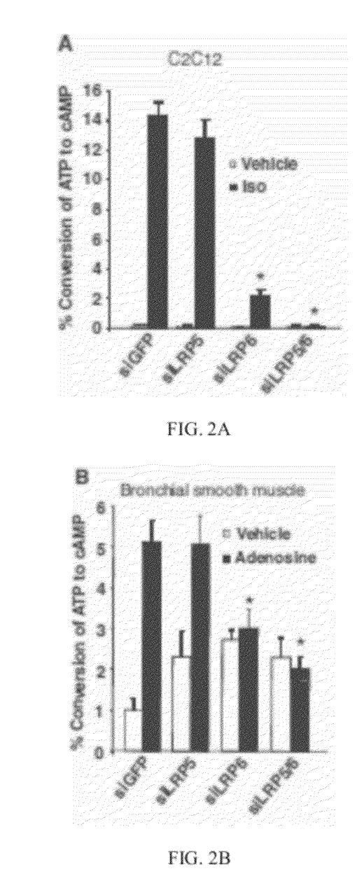

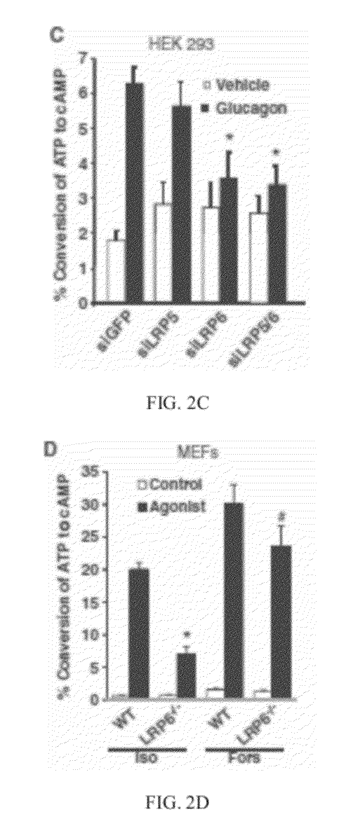

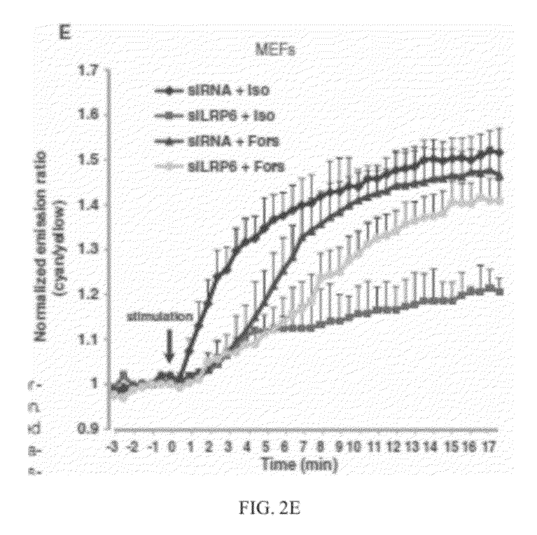

LRP6 is Required for the Generation of cAMP in Response to Different GPCR Ligands. To determine whether LRP6 was also required for the generation of cAMP by other Gs-coupled GPCRs, the effects of LRP6 knockdown on cAMP production were examined in various cell types by different agonists, including by isoproterenol, a β-adrenergic receptor agonist, in C2C12 cells (FIG. 2A), by adenosine in human bronchial smooth muscle cells (FIG. 2B), by glucagon in human embryonic kidney (HEK) 293 cells (FIG. 2C), and by isoproterenol in primary mouse embryo fibroblasts (MEFs) (FIG. 2D). The generation of cAMP was reduced by knockdown of LRP6 in all of these models (FIGS. 2A-2D). The effect of forskolin on the production of cAMP was also inhibited by knockdown of LRP6, but was much less affected than was isoproterenol (FIG. 2D). By monitoring cAMP kinetics in live cells, the knockdown of LRP6 was shown to consistently reduce the responses of these cells to isoproterenol, but only mildly reduced the response of the cells to forskolin (FIG. 2E), indicating that the inhibition of cAMP production that occurred through loss of LRP6 was not primarily through the suppression of AC activity. Together, these data suggest that LRP6 is required in the production of cAMP by Gαₛ-coupled GPCRs.

### Example 3

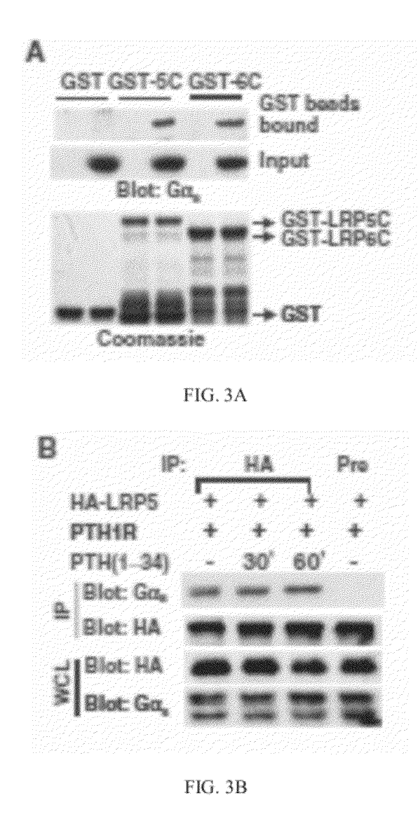

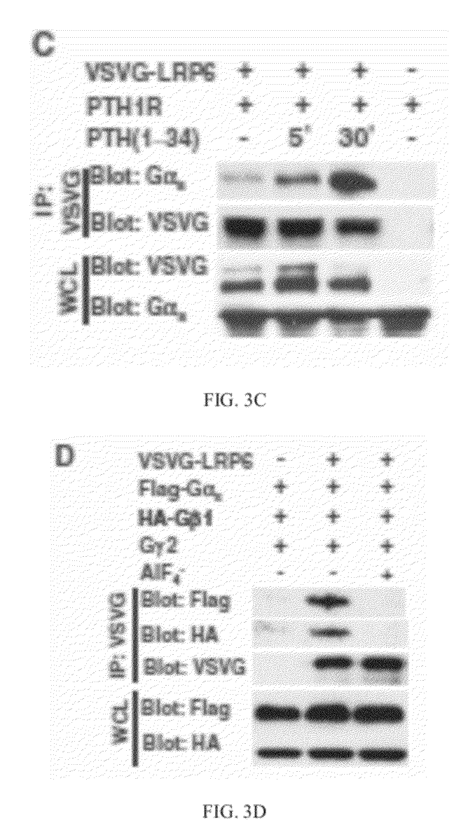

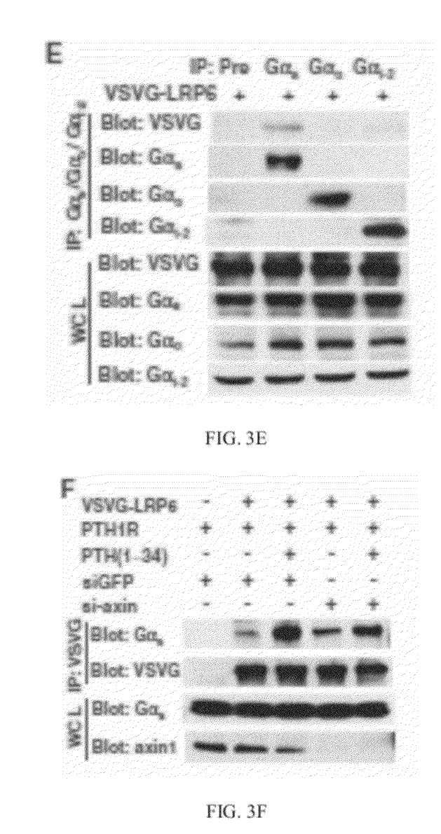

PTH Promotes the Association of LRP6 and Gαs. To determine the mechanisms by which LRP6 was involved in cAMP production induced by Gαₛ-coupled receptors, the interaction of LRP6 with Gαₛ was examined in a pull-down assay. Glutathione S-transferase (GST)-fused cytoplasmic domains of LRP6 or LRP5 were incubated with lysates of HEK 293 cells and it was fund that the intracellular domains of both LRP6 and LRP5 bound to Gαₛ from the cell lysates (FIG. 3A). This was further confirmed in an immunoprecipitation experiment in which HEK 293 cells were transfected with plasmid encoding PTH1R to render them susceptible to stimulation with PTH, together with plasmid encoding hemagglutinin (HA)-tagged LRP5 or vesicular stomatitis virus G (VSVG)-tagged LRP6. Immunoprecipitation followed by Western blotting analysis revealed that Gαₛ formed complexes with both LRP5 (FIG. 3B) and LRP6 (FIG. 3C) in the absence of PTH, and that PTH enhanced the interaction between Gαₛ and LRP6 within S min of treatment and that this association was further enhanced 30 min after addition of the ligand (FIG. 3C). In contrast, PTH did not enhance the association of Gαₛ with LRP5 (FIG. 3B).

Gαₛ and Gβγ firm a heterotrimer on the cell membrane with an inactive configuration. Once activated, Gαₛ is dissociated from βγ dimer. Immunoprecipitations were next performed to examine whether LRP6 interacted with the Gαₛβγ heterotrimer or with the free active form of Gαₛ, and it was found that LRP6 interacted with both Gαₛ and Gβ₁ (FIG. 3D). The interaction of LRP6 with Gαₛ was blocked by AlF₄⁻, a compound that directly activates G protein a subunits, which indicated that the active form of Gαₛ did not interact with LRP6. Moreover, AlF₄⁻ also blocked the interaction of LRP6 with Gβ₁. Thus, LRP6 formed a complex with the inactive configuration of the Gαₛβγ heterotrimer, and the interaction of LRP6 with Gβγ was mediated by the binding of Gαₛ to LRP6. Because the Gαₒ and Gαᵢ proteins are also involved in regulating AC activity, whether LRP6 also formed a complex with these G proteins was examined. It was found that LRP6 interacted with Gαₛ, but had either an undetectable or a weak interaction with Gαₒ and Gαᵢ₂ (FIG. 3E), indicating that LRP6 specifically bound to Gαₛ.

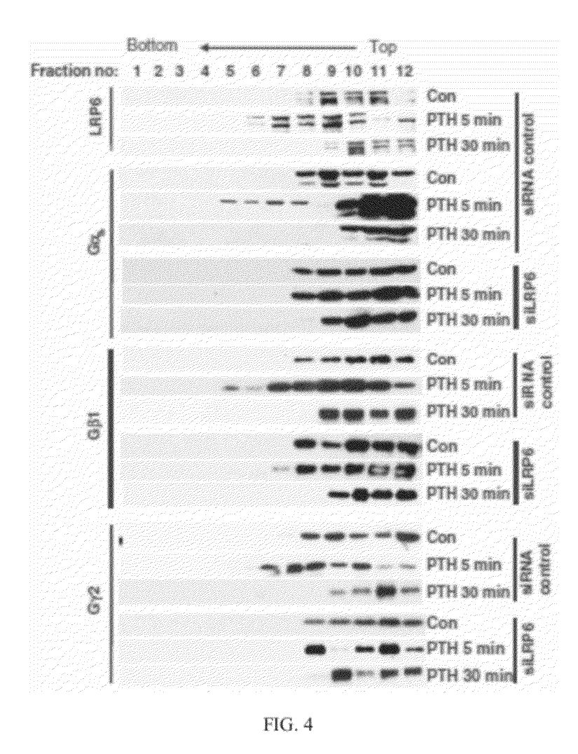

Gαₛ interacts with the b-catenin destruction complex by binding to the scaffolding protein axin (35). Whether axin was involved in the interaction between Gαₛ and LRP6 was examined, but it was found that knockdown of axin did not affect the PTH-induced interaction between LRP6 and Gαₛ (FIG. 3F). Axin binds to the active rather than the inactive form of Gαₛ (35); therefore, these findings suggest that inactive Gαₛ binds to LRP6 and that Gαₛ binds to axin once it is activated. LRP6 aggregates rapidly in response to Wnt3a signaling (45, 46). Because PTH induced the association of Gαₛ with LRP6, the present inventors reasoned that Gαₛβγ might be associated with LRP6 that had aggregated in response to PTH. Sucrose density gradient centrifugation was performed and it was found that LRP6 was detectable in the lower-molecular mass fractions of unstimulated UMR-106 cells (FIG. 4, fractions 8 to 12). LRP6 was redistributed into the heavier fractions of samples that had been treated with PTH for 5 min (FIG. 4, fractions 6 and 7), and this redistribution was lost 30 min after PTH treatment. Gαₛ, Gβ₁, and Cγ₂ were detected in these heavier fractions in response to PTH stimulation. The redistribution of these subunits into the heavier fractions in cells in which LRP6 was knocked down by siRNA was not observed. These results support the concept that PTH stimulated an interaction between LRP6 and the Gαₛβγ heterotrimer and further suggest that LRP6 promoted the redistribution and clustering of Gαₛβγ at the cell membrane in response to PTH. The development of localized high concentrations of Gαₛ could act to enhance the coupling of PTH1Rs to G proteins and play a role in the rapid generation of cAMP that is typical of GPCR stimulation.

### Example 4

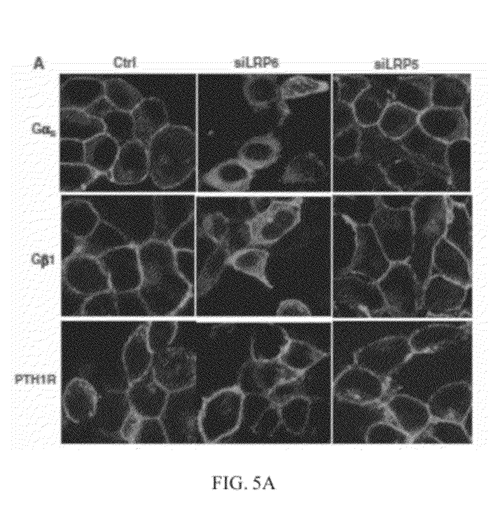

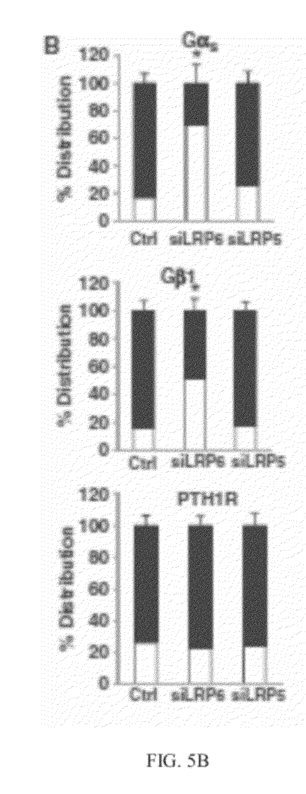

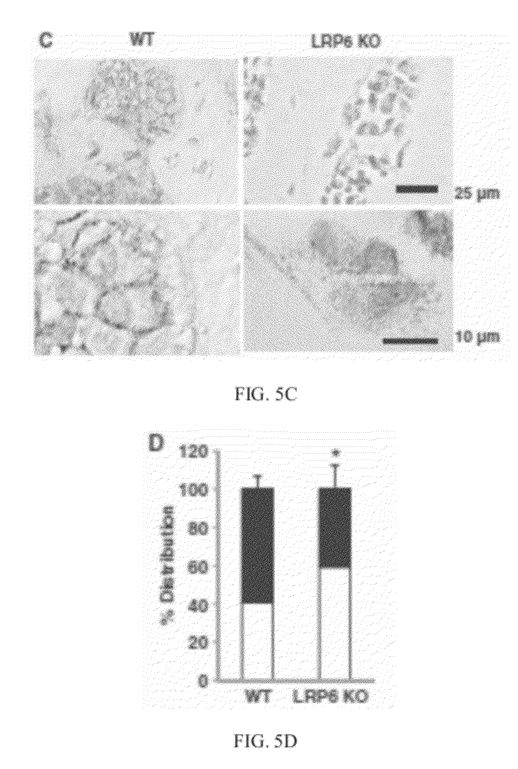

LRP6 Regulates the Localization of Gαs to the Plasma Membrane. It is well established that in the absence of GPCR stimulation, G protein α subunits are in a complex with the β and γ subunits and are associated with the plasma membrane (17-19). These associations are a prerequisite for the coupling of GPCRs with G proteins and the activation of G protein a subunits in response to agonists. Moreover, the localization of G proteins at the cytoplasmic face of the plasma membrane is essential for the coupling of GPCRs to AC. Therefore, whether LRP6 regulated the localization of Gαₛ βγ to the plasma membrane was tested. Both Gαₛ and Gβ₁ were primarily localized to the plasma membrane in control cells and LRP5-deficient cells stably expressing either green fluorescent protein (GFP)-tagged Gαₛ or GFP-tagged Gβ₁ (FIGS. 5A and 5B). A portion of Gαₛ and Gβ₁ was localized to the cytosol when LRP6 was knocked down (FIGS. 5A and 5B). To test the possibility that PTH1R might also undergo trafficking from the plasma membrane to the cytosol in the context of knockdown of LRP6, immunofluorescence analysis of HEK 293 cells stably expressing cyan fluorescent protein (CFP)-tagged PTH1R were performed. PTH1R localized primarily to the plasma membrane, and knockdown of LRP6 did not affect its localization (FIGS. 5A and 5B), indicating that the amount of Gαₛβγ that resided at the plasma membrane and that co-localized with plasma membrane-bound PTH1R was largely reduced when LRP6 was absent.

To confirm this observation in vivo, osteoblast-specific LRP6 knockout mice were generated by crossing lrp6flox/flox mice with OC-Cre mice. Immunohistological analysis of bone sections revealed that Gαₛ was localized primarily at the plasma membrane of osteoblasts in wild-type mice, whereas Gαₛ was found largely in the cytosol of osteoblasts from LRP6 knockout mice (FIGS. 5, C and D). Furthermore, whether LRP6 was also involved in the coupling of PTH1R to Gαₛ was examined, because the present inventors previously demonstrated that PTH induces the formation of a complex between LRP6 and PTH1R (37). The formation of the PTH1R-Gαₛ complex could be estimated by immunoprecipitation of PTH1R from lysates of HEK 293 cells that were stably transfected with a plasmid encoding HA tagged PTH1R followed by Western blotting analysis for Gαₛ, or vice versa. It was found that the amount of the PTH1R-Gαₛ complex was reduced in cells in which LRP6 was knocked down compared to that in control cells (FIGS. 5E and 5F), but that knockdown of LRP5 had no effect (FIG. 5E), suggesting that LRP6 played a role in the formation of this complex. Together, these results indicated that LRP6 regulated the localization to the plasma membrane of a Gαₛ complex to enable its coupling with GPCRs to trigger the production of cAMP.

### Example 5

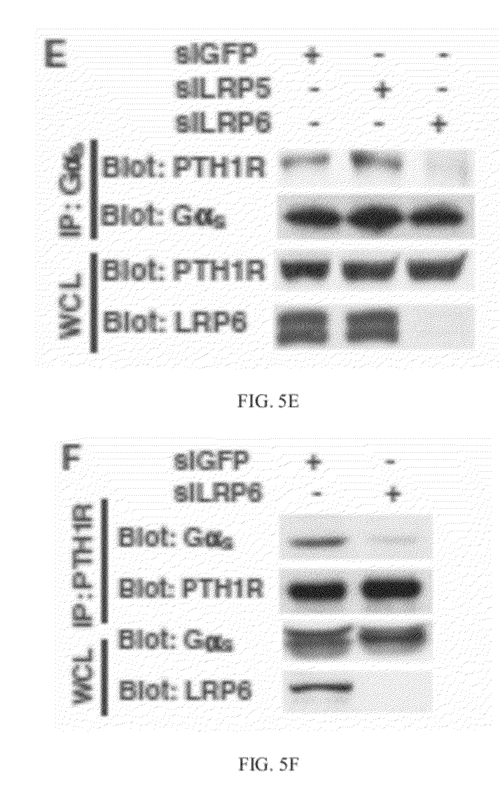

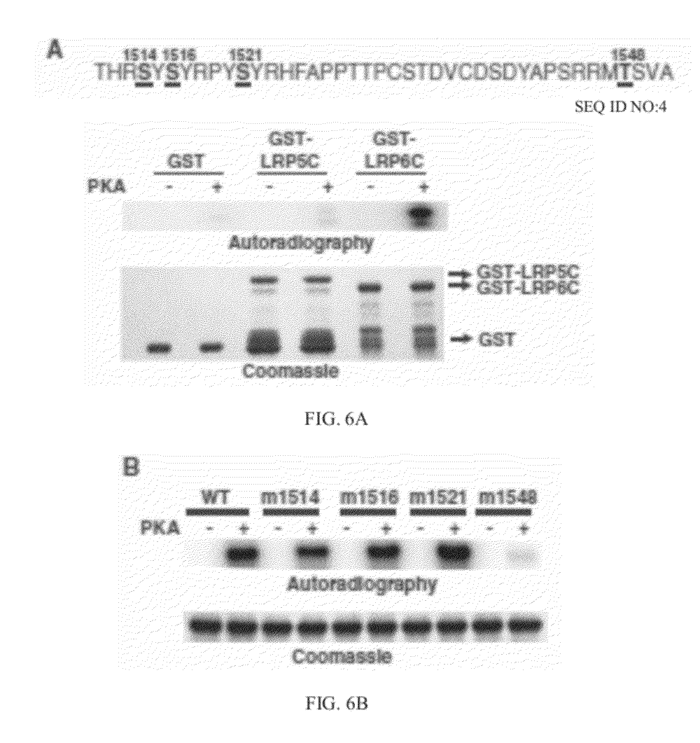

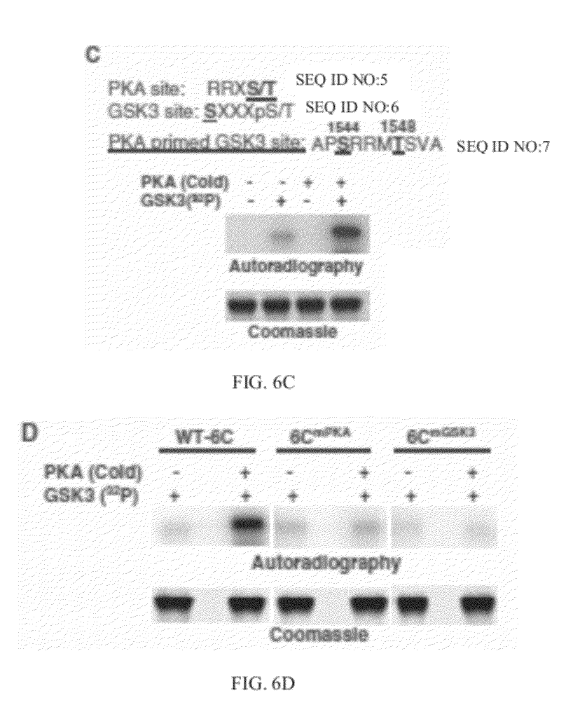

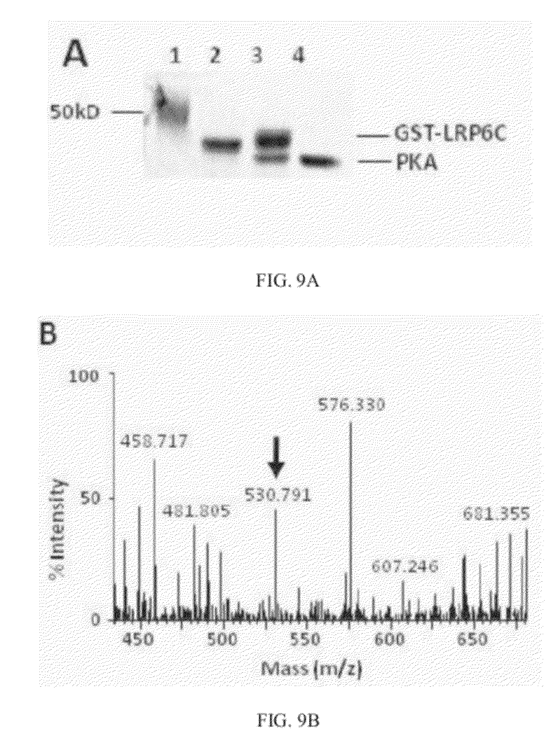

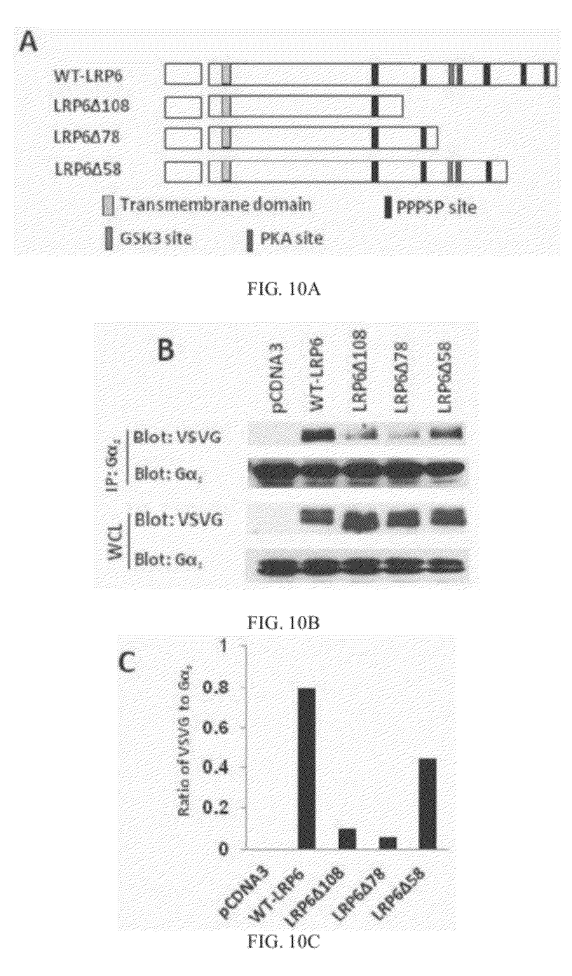

PTH Induces the Phosphorylation of LRP6 at a PKA Target Site. Sequence analysis of the intracellular domain of LRP6 revealed several consensus phosphorylation sites for PKA (RR/K-S*/T* (SEQ ID NO:8); R-X2-S*/T* (SEQ NO:9); and R-X-S*/T* (SEQ ID NO:10)) (FIG. 6A, upper panel) (47). Whether PKA directly phosphorylated LRP6 was therefore examined. In vitro kinase assays demonstrated that the intracellular domain of LRP6 (LRP6C) was phosphorylated by PKA (FIG. 6A, lower panel). In contrast, the intracellular domain of LRP5 was not phosphorylated, which, together with the observation that PTH did not induce the aggregation of LRP5 or promote its interaction with Gαₛ, further suggests that LRP6 plays the predominant role in regulating PTH1R signaling and that phosphorylation of LRP6 by PKA in turn may affect cAMP-PKA signaling. To test this directly, site-directed mutagenesis was first used to modify the individual putative PKA phosphorylation sites in LRP6C. Only alteration of Thr¹⁵⁴⁸ abolished the phosphorylation of LRP6C by PKA (FIG. 6B), and mass spectrometry analysis confirmed that Thr¹⁵⁴⁸ was the PKA phosphorylation site in LRP6C (FIG. 9). HEK 293 cells were then transfected with plasmid encoding the HA-tagged cytoplasmic domain of wild-type LRP6 or a HA-tagged cytoplasmic domain of LRP6 from which the PKA site was deleted (FIG. 10), and the interaction between Gαₛ and LRP6 was analyzed by immunoprecipitation and Western blotting. Deletion of the PKA phosphorylation site (in the mutants LRP6Δ108 or LRP6Δ78) from LRP6 reduced the extent of the interaction with Gαₛ compared to that between wild-type LRP6 and Gαₛ, whereas the addition of this site (LRP6Δ58) rescued, at least partially, the interaction (FIG. 10), which suggested that phosphorylation of the PKA site enhanced the binding of Gαₛ to LRP6. The reason the extent of binding of Gαₛ to the LRP6Δ58 mutant was less than that with wild-type LRP6 (FIG. 10) may be because the basal extent of binding between Gαₛ and LRP6 in the absence of ligand or agonist was independent of the phosphorylation state of LRP6. Removal of 58 amino acid residues from the C terminus of LRP6 might change the conformation of the protein and affect the basal binding to Gαₛ.

GSK-3 is a regulatory kinase that preferentially phosphorylates proteins that have been already phosphorylated at other sites (47). Examination of the amino acid sequence adjacent to Thr¹⁵⁴⁸ in LRP6 revealed a GSK-3 consensus site (Ser¹⁵⁴⁴) (FIG. 6C, upper panel). In an in vitro kinase assay, it was confirmed that pretreatment with recombinant PKA enhanced the phosphorylation of LRP6 by GSK-3 (FIG. 6C, lower panel). Two LRP6C mutants, LRP6CmPKA and LRP6CmGSK3, were generated in which the PKA phosphorylation site (Thr¹⁵⁴⁸ ) or the GSK-3 phosphorylation site (Ser¹⁵⁴⁴) was mutated, respectively. Both mutants abolished the phosphorylation of LRP6 by GSK-3 (FIG. 6D). Thus, these data suggest that LRP6 was sequentially phosphorylated by PKA and GSK-3 at Thr¹⁵⁴⁸ and Ser¹⁵⁴⁴.

### Example 6

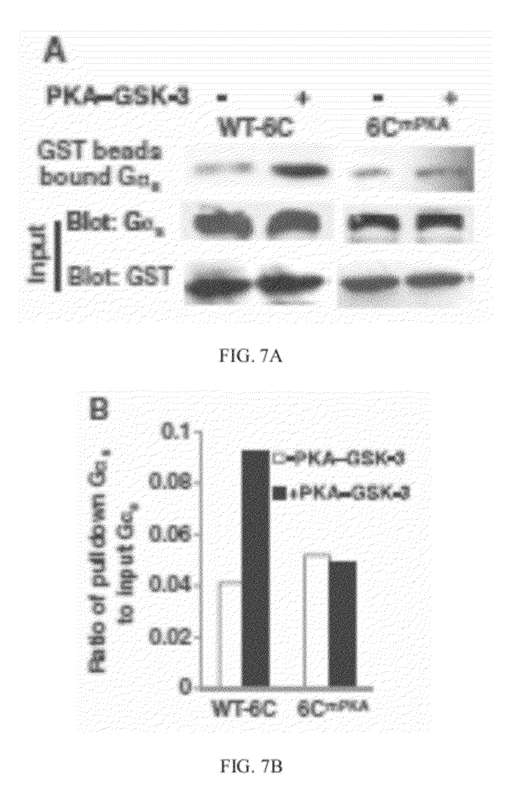

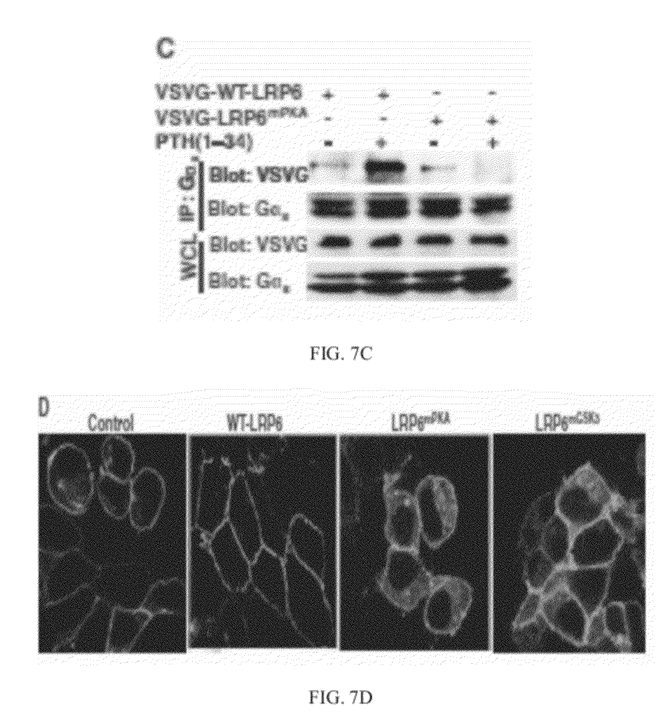

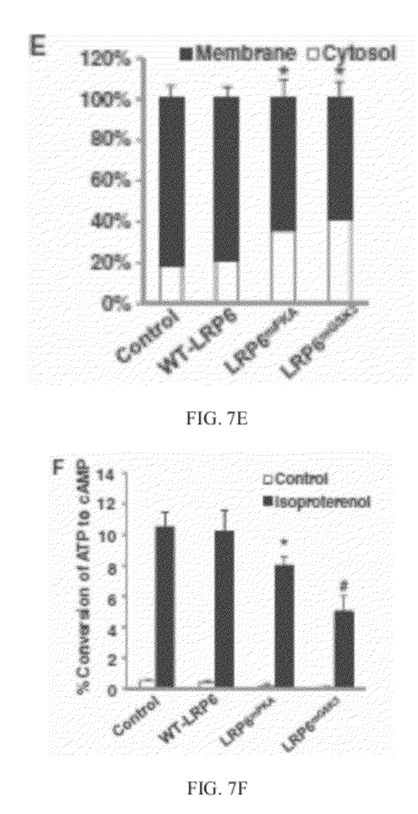

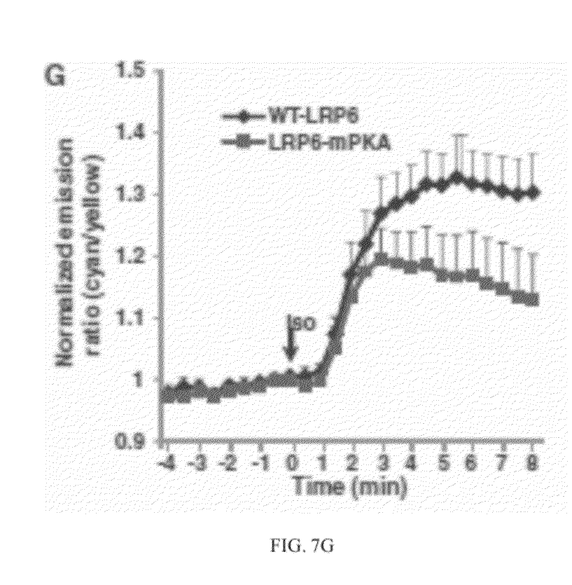

Phoshorylation of LRP6 by PKA Promotes its Binding to Gαs. The role of the PKA site of LRP6 in PTH signaling was next examined. Earlier data provided the framework for further analysis of the effects of the PKA-mediated phosphorylation of LRP 6 on the binding of Gαₛ to LRP6 and on its subcellular location, as well as its role in PTH-induced cAMP production. Sequential phosphorylation of LRP6 by PKA and GSK-3 stimulated the binding of Gαₛ to LRP6C, and mutation of the PKA site abolished the enhanced binding (FIGS. 7A and 7B). Moreover, PTH failed to stimulate the association of Gαₛ with a mutant LRP6 that was missing the PKA phosphorylation site (LRP6^{mPKA}) (FIG. 7C). To test whether the phosphorylation of LRP6 by PKA or GSK-3 regulated the subcellular localization of Gαₛ a stable cell line containing GFR-Gαₛ was transfected with plasmid encoding wild-type LRP6 or LRP6 mutants in which the mutation was at either the PKA(LRP^{mPKA}) or the GSK-3b (LRP6^{mGSK3}) phosphorylation site. Gαₛ was primarily localized to the cell membrane in cells transfected with empty vector or plasmid encoding wild-type LRP6, but a portion of total Gαₛ was translocated to the cytosol in cells that expressed either LRP6^{mPKA} or LRP6^{mGSK3} (FIGS. 7, D and E). As a result, isoproterenol stimulated cAMP production was reduced in cells expressing either mutant compared to that in cells expressing wild-type LRP6 (FIG. 7F). To further examine the role of phosphorylation of LRP6 by PKA in the dynamics of cAMP production, cAMP kinetics were monitored in live cells. The production rate of cAMP at 2 min after isoproterenol stimulation was similar in cells that contained either wild-type LRP6 or LRP6^{mPKA} (FIG. 7G). There was a much smaller response in cells expressing LRP6mPKA compared to that in cells expressing wild-type LRP6 at ˜2 to 8 min after isoproterenol stimulation. Together, these results support the idea that phosphorylation of LRP6 at the PKA site enhances the binding of Gαₛ to LRP6 and accelerates the localization of Gαₛ to the plasma membrane to enable rapid signal amplification.

### Discussion

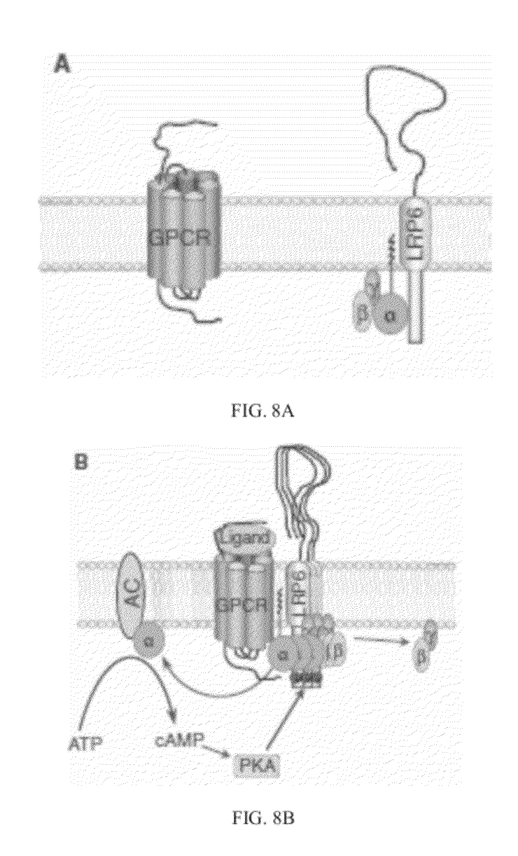

Although studies have shown that LRP6 is involved in the stabilization of b-catenin in response to GPCR stimulation, how LRP6 integrates the signaling of G proteins and b-catenin remains poorly understood. We propose a mechanism for the role of LRP6 in Gαs-coupled, GPCR-stimulated production of cAMP in which LRP6 binds to the Gαₛβγ heterotrimer and the ligands of GPCRs, such as PTH, promote this binding. The interaction between LRP6 and Gαₛβγ induced by PTH would cause the local accumulation of Gαₛβγ at the plasma membrane to set up a functional GPCR-Gαₛβγ-AC complex for the rapid production of cAMP and subsequent PKA activation. Activated PKA would in turn phosphorylate LRP6 at its cytoplasmic domain and enhance its association with Gαₛ and the accumulation of Gαₛβγ to amplify the signal (FIG. 8). Thus, the association of Gαₛ with LRP6 adds another dimension to the regulation of GPCR-mediated cAMP production.

The model that we propose also provides a better understanding of the roles of LRP6 in GPCR-dependent stimulation of two distinct signaling pathways, that is, b-catenin signaling and Gαₛ-cAMP signaling. We identified a specific PKA phosphorylation site (Thr¹⁵⁴⁸) in the cytoplasmic domain of LRP6, phosphorylation of which “primed” the sequential phosphorylation of an adjacent site (Ser¹⁵⁴⁴) by GSK-3. Mutation of these phosphorylation sites inhibited the binding of LRP6 to Gαₛ, disrupted the localization of Gαₛ at the plasma membrane, and reduced the amount of cAMP produced in response to PTH. Therefore, the PKA phosphorylation site within LRP6 is critical for the amplification of Gαₛ signaling.

Several lines of evidence indicate that activation of GPCRs also activates b-catenin signaling (32-37). A study demonstrated that free Gβγ subunits recruited GSK-3β to the plasma membrane to promote the phosphorylation of LRP6 at its PPPS/TP sites and subsequent b-catenin signaling (38), suggesting that free Gβγ is a critical component in GPCR-activated b-catenin signaling. We previously found that PTH induces the phosphorylation of LRP6 at its PPPS/TP sites and that PKA also regulates this phosphorylation event (37). Here, we suggest that the increased binding of Gαₛβγ to PKA-phosphorylated LRP6 accelerates the activation of G proteins, which would result in the increased release of free Gβγ. The enhanced recruitment of GSK-3b to the plasma membrane by free Gβγ and the subsequent phosphorylation of LRP6 at the PPPS/TP sites could be one of the reasons for the involvement of PKA in GPCR-activated b-catenin signaling. The direct binding to axis of the free active form of Gαₛ is also a critical element for the activation of b-catenin signaling (35). Together, this suggests that LRP6 activates Gαs-cAMP signaling and b-catenin signaling sequentially. LRP6 recruits Gαₛβγ to the plasma membrane to accelerate the activation of Gαₛ and its dissociation from Gβγ, and the free Gαₛ and Gβγ subunits promote the activation of b-catenin signaling through two different pathways.

LRP6 interacts with an inactive conformation of Gαₛ⊖γ. In response to PTH, Gαₛ, Gβ₁, and Gγ₂ form aggregates with LRP6 in fractions of the same molecular mass in ultracentrifugation gradients. The interaction of LRP6 with Gαₛβγ was blocked by AlF₄⁻ suggesting that Gαₛ mediated the interaction between LRP6 and the beterotrimer and that LRP6 did not bind to the active form of Gαₛ. In addition, both Gαₛ and _{Gβ1} translocated from the plasma membrane to the cytosol in cells in which LRP6was knocked down, providing further evidence to support the idea that the Gαₛβγ heterotritner, but not the free active form of Gαₛ, was the target of LRP6. The enriched Gαₛβγ heterotrimer on the cell membrane would provide sufficient amounts of the inactive form of Gαₛ, leading to accelerated activation of Gαₛ and the subsequent production of cAMP in response to agonist. We found that the interactions between LRP5 or LRP6 and the Gαₛ complex were different. PTH stimulated the binding of Gαₛ to LRP6, but not LRP5, and PKA phosphorylated LRP6, but not LRP5, in response to PTH. Therefore, the basal interaction of Gαₛ with LRP5 or LRP6 was independent of phosphorylation status, and phosphorylation enhanced the binding of Gαₛ to LRP6.

Gαₛ translocated from the plasma membrane to the cytosol in LRP6-deficient cells and in osteoblasts in the trabecular bone tissue of LRP6 knockout mice. Moreover, mutation of LRP6 at its PKA phosphorylation site reduced the amount of Gαₛ localized to the plasma membrane and increased the cytosolic localization of Gαₛ. Gαₛ is an essential component of the cAMP production system on the plasma membrane. Thus, LRP6-mediated trafficking of Gαₛ to the plasma membrane could set up a functional GPCR-Gαₛ-AC complex for the rapid production of cAMP. Gαₛ is bound to the plasma membrane through a combination of lipid modification and its association with the Gβγ subunits (48). Although a family of palmitoyl acyltransferases (PATS) has been identified (49), the mechanisms by Which Gαₛ is palmitoylated are unclear. Loss of LRP6 might disrupt lipid modification of Gαₛ and indirectly affects its localization to the plasma membrane.

The central role of LRP6 in the Gαₛ-associated production of cAMP in response to GPCR stimulation in mammalian cells has a number of implications. Regulation of the production of cAMP by LRP6 may represent a previously uncharacterized control point that modifies the effects of ligand binding by Gαₛ-associated GPCRs on transcription and other cell functions. This mechanism could potentially integrate hormonal and other Gαₛ-coupled GPCR signals with other extracellular signals. Modulation of cAMP production through agents that target intracellular AC activity is targeted clinically in the treatment of many different diseases (50, 51). Targeting of LRP6 by its agonists or antagonists to modulate Gαₛ activity could provide another potential therapeutic approach.

### REFERENCES

1. L. A. Selbie, S. J. Hill, G protein-coupled-receptor cross-talk: The fine-tuning of multiple receptor-signalling pathways. Trends Pharmacol. Sci. 19, 87-93 (1998).

2. R. S. Ostrom, S. R. Post, P. A. Inset, Stoichiometry and compartmentation in G proteincoupled receptor signaling: Implications for therapeutic interventions involving Gs. J. Pharmacol. Exp. Then 294, 407-412 (2000).

3. R. J. Leficowitz, Seven transmembrane receptors: Something old, something new. Acta Physiol. 190, 9-19 (2007).

4. H. Jüppner, A. B. Abou-Samra, M. Freeman, X. F. Kong, E. Schipani, J. Richards, L. F. Kolakowski Jr., J. Hock, J. T. Potts Jr., H. M. Kronenberg, G. V. Segre, A G protein-linked receptor for parathyroid hormone and parathyroid hormone-related peptide. Science 254, 1024-1026 (1991).

5. A. B, Abou-Samra, H. Jüpper, T. Force, M. W. Freeman, X, F. Kong, E. Schipani, P. Urena, J. Richards, J. V. Bonventre, J. T. Potts Jr., H. M. Kronenberg, G. V. Segre, Expression cloning of a common receptor for parathyroid hormone and parathyroid hormone-related peptide from rat osteoblast-like cells: A single receptor stimulates intracellular accumulation of both cAMP and inositol trisphosphates and increases intracellular free calcium. Proc. Natl. Acad. Sri. U.S.A. 89, 2732-2736 (1992).

6. D. Gesty-Palmer, M. Chen, E. Reiter, S. Ahn, C. D. Nelson, S. Wang, A. E. Eckhardt, C. L. Cowan, R. F. Spurney, L. M. Luttrell, R. J. Lellowitz, Distinct b-arrestin- and protein-dependent pathways for parathyroid hormone receptor-stimulated ERK1/2 activation. J. Biol. Chem. 281, 10856-10864 (2006).

7. S. G. Rasmussen, H. J. Choi, D. M. Rosenbaum, T. S. Kobilka, F. S. Thian, P. C. Edwards, M. Burghammer, V. R. Ratnaia, R. Sanishvili, R. F. Fischetti, G. F. Schertier, W. I. Weis, B. K. Kobilka, Crystal structure of the human b2 adrenergic G-protein-coupled receptor. Nature 450, 383-387 (2007).

8. T. Huber, S. Menon, T. P. Sakmar, Structural basis for ligand binding and specificity in adrenergic receptors: Implications for GPCR-targeted drug discovery. Biochemistry 47, 11013-11023 (2008).

9. N. M. Sherwood, S. L. Krueckl, J. F. McRory, The origin and function of the pituitary adenylate cyclase-activating polypeptide (PACAP)/glucagon superfamily. Endocr. Rev. 21, 619-670 (2000).

10. R. W. Gelling, X. Q. Du, D. S. Dichmann, J. Romer, H. Huang, L. Cui, S. Obici, B, Tang, J. J. Hoist, C. Fledelius, P. B. Johansen, L. Rossetti, L. A. Jelicks, P. Serup, E. Nishimura, M. J. Charron, Lower blood glucose, hyperglucagonemia, and pancreatic a cell hyperplasia glucagon receptor knockout mice. Proc. Natl. Acad. Sci. U.S.A. 100, 1438-1443 (2003).

S. J. Lolait, A. M. O'Carroll, O. W. McBride, M. Konig, A. Morel, M. J. Brownstein, Cloning and characterization of a vasopressin V2 receptor and possible link to nephrogenic diabetes insipidus. Nature 357, 336-339 (1992).

12. L. S. Weinstein, S. Yu C. A. Ecelbarger, Variable imprinting of e heterotrimeric G protein Gsa-subunit within different segments of the nephron. Am. J. Physiol. Renal Physiol. 278, F507-F514 (2000).

13. K. G. Mountjoy, L. S. Robbins, M. T. Mortrud, R. D. Cone, The cloning of a family of genes that encode the melanocortin receptors. Science 257, 1248-1251 (1992).

14. S. J. Getting, Targeting melanocortin receptors as potential novel therapeutics. Pharmacol. Ther. 111, 1-15 (2006).

15. P. C. Leung, J. Wang, The role of inositol lipid metabolism in the ovary. Biol. Reprod. 40, 703-708 (1989).

16. M. Ascoli, F. Fanelli, D. L. Segaloff, The lutropin/choriogonadotropin receptor, a 2002 perspective. Endocr. Rev. 23. 141-174 (2002).

17. M. E. Linder, P. Middleton, J. R. Hepler, R. Taussig, A. G. Gilman, S. M. Mumby, Lipid modifications of G proteins: a subunits are palmitoylated Proc. Natl. Acad. Sci. U.S.A. 90, 3675-3679 (1993).

18. P. B. Wedegaertner, H. R. Bourne, Activation and depalmitoylation of Gsa. Cell 77, 1063-1070 (1994).

19. C. Kleuss, E. Krause, Gas is palmitoylated at the N-terminal glycine. EMBO J. 22. 826-832 (2003).

20. S. M. Mumby, P. J. Casey, A. G. Gilman, S. Gutowski, P. C. Sternweis, G protein g subunits contain a 20-carbon isoprenoid. Proc. Natl. Acad. Sci. U.S.A. 87, 5873-5877 (1990).

21. R. K. Lai, D. Perez-Sala, F. J. Cañada., R. R. Rando, The g subunit of transducin is farnesylated. Proc. Natl. Acad. Sci. U.S.A. 87, 7673-7677 (1990).

22. Y. Marrari, M. Crouthamel, R. Irannejad, P. B. Wedegaertner, Assembly and trafficking of heterotrimeric G proteins. Biochemistry 46, 7665-7677 (2007).

23. X. He, M. Semenov, K. Tamai, X. Zeng, LDL receptor-related proteins 5 and 6 in Wnt/b-catenin signaling: Arrows point the way. Development 131, 1663-1677 (2004).

24. B. O. Williams, K. L. Insogna, Where Wnts went: The exploding field of Lrp5 and Lrp6 signaling in bone. J. Bone Miner. Res. 24, 171-178 (2009).

25. K. I. Pinson, J. Brennan, S. Monkley, B. J. Avery, W. C. Skarnes, An LDL-receptorrelated protein mediates Wnt signalling in mice. Nature 407, 535-538 (2000).

26. K. Tamai, M. Semenov, Y. Kato, R. Spokony, C. Liu, Y. Katsuyama, F. Hess, J. P. Saint-Jeannet, X. He, LDL-receptor-related proteins in Wnt signal transduction. Nature 407, 530-535 (2000).

27. B. Mao, W. Wu, Y. Li, D. Hoppe, P. Stannek, A. Glinka, C. Niehrs, LDL-receptor-related protein 6 is a receptor for Dickkopf proteins. Nature 411, 321-325 (2001).

28. A. Bafico, G, Liu, A. Yaniv, A. Gazit, S. A, Aaronson, Novel mechanism of Wnt signalling inhibition mediated by Dickkopf-1 interaction with LRP6/Arrow. Nat. Cell Biol. 3, 683-686 (2001).

29. M. Sëmenov, K. Tamai, X. He, SOST is a ligand for LRP5/LRP6 and a Wnt signaling inhibitor. J. Biol. Chem. 280, 26770-26775 (2005).

30. D. L. Ellies, B. Viviano, J. McCarthy, J. P. Rey, N. Itasaki, S. Saunders, R. Krumlauf, Bone density ligand, Sclerostin, directly interacts with LRP5 but not LRP5G171V to modulate Wnt activity. J. Bone Miner. Res. 21, 1738-1749 (2006).

31. X. Li, Y. Zhang, H. Kang, W. Liu, P. Liu, J. Zhang, S. E. Harris, D. Wu, Sclerostin binds to LRP5/6 and antagonizes canonical Wnt signaling. J. Biol. Chem. 280, 19883-19887 (2005).

32. H. Fujino, D. Srinivasan, J. W. Regan, Cellular conditioning and activation of b-catenin signaling by the FPB prostanoid receptor. J. Biol. Chem. 277, 48786-48795 (2002).

33. G. G. Faris, J. A. Godoy, F. Hernández, J. Avila, A. Fisher, N. C. Inestrosa, M1 muscarinic receptor activation protects neurons from b-amyloid toxicity. A role for Wnt signaling pathway. Neurobiol. Dis. 17, 337-348 (2004).

34. M. Yang, W. W. Zhong, N. Srivastava, A. Slavin, J. Yang, T. Hoey, S. An, G proteincoupled lysophosphatidic acid receptors stimulate proliferation of colon cancer cells through the b-catenin pathway. Proc. Natl. Acad. Sci. U.S.A. 102, 6027-6032 (2005).

35. M. D. Castellone, H. Teramoto, B. O. Williams, K. M. Druey, J. S. Gutkind, Prostaglandin E2 promotes colon cancer cell growth through a Gs-axin-b-catenin signaling axis. Science 310, 1504-1510 (2005).

36. S. Gardner, S. Maudsley, R. P. Millar, A. J. Pawson, Nuclear stabilization of b-catenin and inactivation of glycogen synthase kinase-3b by gonadotropin-releasing hormone: Targeting Wnt signaling in the pituitary gonadotrope. Mol. Endocrinol. 21, 3028-3038 (2007).

37. M. Wan, C. Yang, J. Li, X. Wu, H. Yuan, H. Ma, X. He, S. Nie, C. Chang, X. Cao, Parathyroid hormone signaling through low-density lipoprotein-related protein 6. Genes Dev. 22, 2968-2979 (2008).

38. K. K. Jernigan, C. S. Cselenyi, C. A. Thorne, A. J. Hanson, E. Tahinci, N. Hajicek, W. M. Oldham, L. A. Lee, H. E. Hamm, J. R. Hepler, T. Kozasa, M. E. Linder, E. Lee, Gbg activates GSK3 to promote LRP6-mediated b-catenin transcriptional activity. Sci. Signal. 3, ra37 (2010).

39. J. T. Potts Jr., T. M. Murray, M. Peacock, H. D. Niall, G. W. Tregear, H. T. Keutmann, D. Powell, F. J. Deftos, Parathyroid hormone: Sequence, synthesis, immunoassay studies. Am. J. Med. 50, 639-649 (1971).

40. C. R. Zylstra, C. Wan, K. K. VanKoevering, A. K. Sanders, C. Lindvall, T. L. Clemens, B. O. Williams, Gene targeting approaches in mice: Assessing the roles of LRP5 and LRP6 in osteoblasts. J. Musculoskelet. Neuronal Interact. 8, 291-293 (2008).

41. L. M. DiPilato, X. Cheng, J. Zhang, Fluorescent indicators of cAMP and Epac activation reveal differential dynamics of cAMP signaling within discrete subcellular compartments. Proc. Natl. Acad. Sci. U.S.A. 101, 16513-16518 (2004).

42. J. D. Violin, L. M. DiPilato, N. Yaldirim, T. C, Elston, J. Zhang, R. J. Letkowitz, b2-Adrenergic receptor signaling and desensitization elucidated by quantitative modeling of real time cAMP dynamics. J. Biol. Chem. 283, 2949-2961 (2008).

43. L. M. DiPilato, J. Zhang, The role of membrane microdomains in shaping b2-adrenergic receptor-mediated cAMP dynamics. Mol. Biosyst. 5, 832-837 (2009).

44. K. B. Seamon, W. Padgett, J. W. Daly, Forskolin: Unique diterpene activator of adenylate cyclase in membranes and in intact cells. Proc. Natl. Acad. Sci. U.S.A. 78, 3363-3367 (1981).

45. J. Bilic, Y. L. Huang, G. Davidson, T. Zimmermann, C. M. Crucial M. Bienz, C. Niehrs, Wnt induces LRP6 signalosomes and promotes Dishevelled-dependent LRP6 phosphorylation. Science 316, 1619-1622 (2007).

46. W. Pan, S. C. Choi, H. Wang, Y, Qin, F. Volpicelli-Daley, L. Swan, L. Lucast, C. Khoo, X. Zhang, L. Li, C. S. Abrams, S. Y. Sokol, D. Wu, Wnt3a-mediated formation of phosphatidylinositol 4,5-bisphosphate regulates LRP6 phosphorylation. Science 321, 1350-1353 (2008).

47. C. J. Fiol, A. Wang, R. W. Roeske, P. J. Roach, Ordered multisite protein phosphorylation. Analysis of glycogen synthase kinase 3 action using model peptide substrates. J. Biol. Chem. 265, 6061-6065 (1990).

48. M. D. Resh, Palmitoylation of ligands, receptors, and intracellular signaling molecules. Sci. STKE 2006, rel4 (2006).

49. R. Tsutsutni, Y. Fukata, M. Fukata, Discovery of protein-palmitoylating enzymes. Pflugers Arch. 456, 1199-1206 (2008).

50. S. Pierre. T. Eschenhagen, G. Geisslinger, K. Scholich. Capturing adenylyl cyclases as potential drug targets. Nat. Rev. Drug Discov. 8, 321-335 (2009).

51. B. Pavan, C. Biondi, A. Dalpiaz, Adenylyl cyclases as innovative therapeutic goals. Drug Discov. Today 14, 982-991 (2009).

52. E. Schipani, H. Karga, A. C. Karaplis, J. T. Potts Jr., H. M. Kronenberg, G. V. Segre, A. B. Abou-Samra, H. Jüppner, Identical complementary deoxyribonucleic acids encode a human renal and bone parathyroid hormone (PTH)/PTH-related peptide receptor. Endocrinology 132, 2157-2165 (1993).

53. J. Z. Yu, M. M. Rasenick, Real-time visualization of a fluorescent Gas: Dissociation of the activated G protein from plasma membrane. Mol. Pharmacol. 61, 352-359 (2002).

54. S. M. Kim, E. J. Choi, K. J. Song, S. Kim, E. Seo, E. H. Jho, S. H. Kee, Axin localizes to mitotic spindles and centrosomes in mitotic cells. Exp. Cell Res. 315, 943-954 (2009).

55. N. Gallo-Payet, M. D. Payet, Excitation-secretion coupling: Involvement of potassium channels in ACTH-stimulated rat adrenocortical cells. J. Endocrinol. 120, 409-421 (1989).

56. Z. Kilianova, N. Basora, P. Kilian, M. D. Payet, N. Gallo-Payet, Human melanocortin receptor 2 expression and functionality: Effects of protein kinase A and protein kinase Con desensitization and internalization. Endocrinology 147, 2325-2337 (2006).

57. Y. Salomon, C. Londos, M. Rodbell, A highly sensitive adenylate cyclase assay. Anal. Biochem. 58, 541-548 (1974).

58. M. Wan, X. Cao, Y. Wu, S. Bai, L. Wu, X. Shi, N. Wang, X. Cao, Jab1 antagonizes TGF-b signaling by inducing Smad4 degradation. EMBO Rep. 3, 171-176 (2002).

59. C. J. Doering, A. E. Kisilevsky, Z. P. Feng, M. I. Arnot, J. Peloquin, J. Hamid, W. Barr, A. Nirdosh, B. Simms, R. J. Winkfein, O. W. Zamponi, A single Gb subunit locus controls cross-talk between protein kinase C and G protein regulation of N-type calcium channels, J. Biol. Chem. 279, 29709-29717 (2004).

60. F. Zhang, T. Qiu, X. Wu, C. Wan, W. Shi, Y. Wang, J. G. Chen, M. Wan, T. L. Clemens, X. Cao, Sustained BMP signaling in osteoblasts stimulates bone formation by promoting angiogenesis and osteoblast differentiation. J. Bone Miner. Res. 24, 1224-1233 (2009).

61. Y. Tang, X. Wu, W. Lei, L. Pang, C. Wan, Z. Shi, L. Zhao, T. R. Nagy, X. Peng, J. Hu, X. Feng, W. Van Hul, M. Wan, X. Cao, TGF-b1-induced migration of bone mesenchymal stem cells couples bone resorption with formation. Nat. Med. 15, 757-765 (2009).

We claim:

1. A method of screening for antagonists of LRP6-Gαₛ interaction comprising the steps of:
a. contacting an agent that binds LRP6 with a cell; and
b. measuring the level of cAMP production in the cell in response to one or more GPCR ligands, wherein an agent that decreases cAMP production as compared to cAMP production in the cell not contacted with the agent identifies the agent as an antagonist of LRP6-Gαₛ interaction.

2. The method of claim 1, wherein Gαₛ is complexed with Gβγ in a heterotrimer.

3. The method of claim 1, wherein the GPCR ligand is PTH(1-34) and the cell is an osteosarcoma cell.

4. The method of claim 1, wherein the GPCR ligand is isoproterenol and the cell is an osteoprogenitor cell.

5. The method of claim 1, wherein the GPCR ligand is adenosine and the cell is a bronchial smooth muscle cell.

6. The method of claim 1, wherein the GPCR ligand s glucagon and the cell is an embryonic kidney cell.

7. The method of claim 1, wherein the GPCR ligand is isoproterenol and the cell is an embryo fibroblast.

8. The method of claim 1, further comprising contacting the cell with forskolin.

9. The method of claim 1, wherein the agent is a small molecule, an antibody, polypeptide, a polynucleotide, an aptamer, or an siRNA.

10. A method for treating an LRP6-mediated disease comprising the step of administering to a patient an effective amount of the antagonist of claim 1.

11. A method for identifying an LRP6 modulator comprising the step of performing a kinase assay using LRP6 and cAMP-dependent protein kinase (PKA) in vitro in the presence and absence of a test agent, wherein an agent that inhibits or reduces the phosphorylation of LRP6 by PKA is identified as an LRP6 modulator.

12. The method of claim 11, wherein the agent is a small molecule, an antibody, polypeptide, a polynucleotide, an aptamer, or an siRNA.

13. A method for treating an LRP6-mediated disease comprising the step of administering to a patient an effective amount of the LRP6 modulator of claim 11.

14. A method of screening for LRP6 modulators comprising the steps of:
a. contacting a cell that expresses LRP6 with an agent;
b. assaying LRP6 activity; and
c. comparing the assayed LRP6 activity to LRP6 activity in cell that has not been contacted with the agent, wherein a difference in the compared LRP6 activities identifies the agent as an LRP6 modulator.

15. The method of claim 14, wherein the LRP6 activity is determined by measuring the level of LRP6 mRNA, measuring the level of β-catenin, or measuring the level of cAMP production.

16. The method of claim 14, wherein the agent is a small molecule, an antibody, polypeptide, a polynucleotide, an aptamer, or an siRNA.

17. A method for treating an LRP6-mediated disease comprising the step of administering to a patient an effective amount of the antagonist of claim 14.

18. A method of screening for therapeutic agents useful in the treatment of LRP6-mediated diseases comprising the steps of:
a. contacting a test agent with an LRP6 polypeptide; and
b. detecting the binding of the test agent to the LRP6 polypeptide.

19. The method of claim 18, further comprising:
c. contacting the test agent with a cell derived from a patient suffering from an LRP6-mediated disease; and
d. determining the effect of the test agent on the cell.

20. The method of claim 19, wherein the agent is a small molecule, an antibody, polypeptide, a polynucleotide, an aptamer, or an siRNA.

21. A method for treating an LRP6-mediated disease comprising the step of administering to a patient an effective amount of the antagonist of claim 19.
